SEQUENCE LISTING (49 sequences)

SEQ ID NO: 1 - DNA, 11 bp, Mus musculus
gaaaacctgga

SEQ ID NO: 2 - DNA, 14 bp, Homo sapiens
agaaagtgaagtga

SEQ ID NO: 3 - DNA, 11 bp, Mus musculus
cgtatcgaaac

SEQ ID NO: 4 - DNA, 15 bp, Homo sapiens
ccgaaaccgaaacta

SEQ ID NO: 5 - DNA, 17 bp, Homo sapiens
aatgccacgtcatcatc

SEQ ID NO: 6 - RNA, 20 nt, Homo sapiens
ggaaagcccugggcucggcc

SEQ ID NO: 7 - RNA, 20 nt, Homo sapiens
ccccacuucgcgcagccgag

SEQ ID NO: 8 - RNA, 20 nt, Homo sapiens
augcgaagagaggaagcucu

SEQ ID NO: 9 - RNA, 20 nt, Homo sapiens
cgggaaccccaccccggccg

SEQ ID NO: 10 - RNA, 20 nt, Homo sapiens
ccucuccccaguccaacccc

SEQ ID NO: 11 - RNA, 20 nt, Homo sapiens
gcagcccccagccuucacgc

SEQ ID NO: 12 - RNA, 20 nt, Homo sapiens
ggcguggcagcggcaauccc

SEQ ID NO: 13 - RNA, 20 nt, Homo sapiens
aggaccguggcuauggaguc

SEQ ID NO: 14 - RNA, 20 nt, Homo sapiens
cggccgagcucggcguccau

SEQ ID NO: 15 - RNA, 20 nt, Cricetulus griseus
auaguuggaagugugcugac

SEQ ID NO: 16 - RNA, 20 nt, Cricetulus griseus
cuuucucuaacuaggcaaug

SEQ ID NO: 17 - RNA, 20 nt, Cricetulus griseus
uugccuguguuuguucugag

SEQ ID NO: 18 - RNA, 20 nt, Cricetulus griseus
ugcagaguucggcaugagcg

SEQ ID NO: 19 - RNA, 20 nt, Cricetulus griseus
cugaucgaccagaucgacag

SEQ ID NO: 20 - RNA, 20 nt, Cricetulus griseus
gauuacacaaacagacaacc

SEQ ID NO: 21 - RNA, 20 nt, Cricetulus griseus
uucagugagccaacucucug

SEQ ID NO: 22 - RNA, 20 nt, Cricetulus griseus
gguggcagcgucgccgagcg

SEQ ID NO: 23 - RNA, 20 nt, Cricetulus griseus
cgauagaagcaguacuuucg

SEQ ID NO: 24 - RNA, 20 nt, Cricetulus griseus
uuccaggcucgcgggcagca

SEQ ID NO: 25 - RNA, 20 nt, Cricetulus griseus
ccucacgcaccugagcccgg

SEQ ID NO: 26 - RNA, 20 nt, Cricetulus griseus
gcguuacucccacaggugag

SEQ ID NO: 27 - RNA, 20 nt, Cricetulus griseus
aacgcggucagucagagccg

SEQ ID NO: 28 - RNA, 20 nt, Cricetulus griseus
ggcuucuggcgugugaccgg

SEQ ID NO: 29 - RNA, 20 nt, Cricetulus griseus
cgaaggcagccguccccccg

SEQ ID NO: 30 - RNA, 20 nt, Cricetulus griseus
ggcugcgugaaagccuugag

SEQ ID NO: 31 - RNA, 20 nt, Cricetulus griseus
aagcgcuaauuacagcccgg

SEQ ID NO: 32 - RNA, 20 nt, Cricetulus griseus
cuuaucaugucugcucgaag

SEQ ID NO: 33 - RNA, 20 nt, Cricetulus griseus
ucaugucugcucgaagcggc

SEQ ID NO: 34 - RNA, 20 nt, Cricetulus griseus
ccacuuuguacaagaaagcu

SEQ ID NO: 35 - RNA, 20 nt, Cricetulus griseus
uuuuauguuucagguucagg

SEQ ID NO: 36 - RNA, 20 nt, Cricetulus griseus
cuccuccucuugguacgccc

SEQ ID NO: 37 - RNA, 20 nt, Cricetulus griseus
agaggaggagccggaucuuc

SEQ ID NO: 38 - RNA, 20 nt, Cricetulus griseus
ggugccaccagauucgcacg

SEQ ID NO: 39 - RNA, 20 nt, Cricetulus griseus
aaucacguacugcagccagg

SEQ ID NO: 40 - RNA, 20 nt, Cricetulus griseus
ggccaccgagaaucggacgg

SEQ ID NO: 41 - RNA, 20 nt, Cricetulus griseus
acacagguaagugccgugug

SEQ ID NO: 42 - RNA, 20 nt, Cricetulus griseus
gcgggccaagaucugcacac

SEQ ID NO: 43 - RNA, 20 nt, Cricetulus griseus
aaaagcucgagaacuaaucg

SEQ ID NO: 44 - RNA, 20 nt, Cricetulus griseus
gccaccagauucgcacgcgg

SEQ ID NO: 45 - RNA, 20 nt, Cricetulus griseus
uacccccguccgauucucgg

SEQ ID NO: 46 - RNA, 20 nt, Cricetulus griseus
ggacggcgcccgguacuccg

SEQ ID NO: 47 - RNA, 20 nt, Cricetulus griseus
cccagcgcacauguucggcg

SEQ ID NO: 48 - RNA, 20 nt, Cricetulus griseus
accugggacgacucccugaa

SEQ ID NO: 49 - RNA, 20 nt, Cricetulus griseus
gccguucagggagucguccc

(19) United States
(12) Patent Application Publication
(10) Pub. No.: US 2024/0271127 A1
(43) Pub. Date: Aug. 15, 2024

(54) METHODS OF ENHANCING AND EXPEDITING EXPRESSION OF ANTIBODIES

(71) Applicant: Aridis Pharmaceuticals, Inc., Los Gatos, CA (US)

(72) Inventors: Kathy Ngo, San Jose, CA (US); Jennifer Woo, La Mesa, CA (US); Binh Pham, Mountain View, CA (US); Vu Truong-Le, Campbell, CA (US)

(21) Appl. No.: 17/784,796

(22) PCT Filed: Dec. 15, 2020
(86) PCT No.: PCT/US20/65054

§ 371 Date: Jun. 13, 2022

Related U.S. Application Data
(60) Provisional application No. 62/949,422, filed on Dec. 17, 2019.

(51) Int. Cl. C12N 15/11; C07K 16/00; C12N 5/071; C12N 9/22

(52) U.S. Cl. CPC C12N 15/111; C07K 16/00; C12N 5/0682; C12N 9/22; C07K 2317/14; C12N 2310/20

(57) ABSTRACT

This application describes cells, systems, and molecular engineering methods using CRISPR/Cas complexes for targeted activation of endogenous master transcriptional regulatory elements (MTRE) such as PRDM1, XBP1 and IRF4, to generate high productivity antibody production in production cell lines such as CHO and NSO cells. These incorporate the inclusion of the Cas accessory proteins, design of multiple guide RNAs (gRNA), and unique multiplexing of these components using, e.g., lentiviral transfection to induce increased transcription and translation of antibody genes under the control of the MTRE. The methods result in synergies increasing monoclonal antibody production by these modified cell lines. While a significant increase in productivity is demonstrated by this method of activation, further increase in productivity can be accomplished by genetic transfer of additional copies of MTREs.

### FIELD OF THE INVENTION

Engineered elements can be incorporated in master production cell lines with known production levels and expressed defined monoclonal antibody (mAb) components using this described method that facilitates the exchange of either the heavy and light chain genes or the complementarity determining region (CDR) domains in order to leverage the improved antibody production capability of the master cell lines in the generation of new production cell lines expressing antibodies specified by the exchanged gene products. These described Methods to increase antibody production will include inter alia the triplex activation and gene amplification of endogenous master transcriptional regulatory elements. The concomitant integration of multiple elements at the transcriptional, post-transcriptional, translational and post-translational level combined with site-specific gene product exchange provides synergistic antibody expression enhancement in production cell lines while reducing the variability for manufacturing process development.

### BACKGROUND OF THE INVENTION

There is need for rapid production of mAb at high titer for clinical and commercial utilization. One method for production of monoclonal antibodies (mAbs) is the generation of hybridoma cells which is well-known in the art. The methods used to produce monoclonal antibodies are disclosed by Kohler and Milstein in Nature 256, 495-497 (1975) and also by Donillard and Hoffman, “Basic Facts about Hybridomas” in Compendium of Immunology V. II ed. by Schwartz, 1981. Human hybridomas are obtained by fusion of a human B cell with a myeloma or heteromyeloma cell. The production process comprises culturing a hybridoma under conditions allowing for secretion of an antibody, and purifying the antibody from the culture supernatant. The generation of mAbs for therapeutic use requires mAbs to be manufactured and produced at scale using cells with key manufacturing characteristics including stability of production and high titer productivity.

An alternative method to hybridoma technology is the recombinant expression of nucleic acids encoding the antibody light and heavy chain in mammalian cell lines. Mammalian cell lines are preferred as they offer the advantage of generating transcriptional, translational, and post-translational modifications most similar to human cells. The most commonly used mammalian cell lines are Chinese hamster ovary cells (CHO), murine myeloma cell line NS0 (European Collection of Animal Cell Cultures, ECACC number 85110503), and human embryonic kidney 293 cells (HEK293). However, the generation of such cell lines for efficient antibody manufacturing and production requires a 1.5 to 2-year commitment due to the variability of each cell line.

Cell line engineering is a method to improve cell lines and recent discoveries and advances in gene editing technologies such as ZFNs (zinc finger nucleases), TALENs (transcription activator-like effector nucleases) and the CRISPR/Cas system enable efficient and directed cell engineering. Current approaches have been used in the regulation of apoptosis, metabolic engineering, engineering cells for growth at lower temperature, chaperone engineering and glyco-engineering (Dangi et al, 2018).

Of specific interest is the use of CRISPR/Cas genes which were discovered as a natural defense mechanism in bacteria used for the control of pathogens. Recent studies have expanded their application as tools for use in engineering eukaryotic cells.

In view of the above, a need exists to eliminate redundant efforts involved in the development and validation of antibody production processes. Benefits can be realized through methods to enhance the quantity of antibody secretion from host cells in culture. The present inventions provide these and other features that will be apparent upon review of the following.

### SUMMARY OF THE INVENTION

The present inventions are directed to cells, systems, and methods for enhancing and simplifying expression of antibodies, e.g., through a synergistic interaction of novel enhancement elements with endogenous features of expression host cell lines.

Host cells can initially be expressing (e.g., transcribing, translating, and secreting) a given antibody. Non-endogenous tools can be introduced into the cell to cooperate synergistically providing many-fold increases in antibody production over standard host cell expression. Further, well characterized host cell lines producing one antibody product can be rapidly modified to produce an antibody to a different target, while retaining familiar validated attributes.

Described herein is CRISPR/dCas9 linked to transcription activators capable of obtaining greater than endogenous levels of expression. In many embodiments, the transcription activators are connected to the C-terminus of dCas9 and activate expression of transcription factors, which in turn stimulate expression of proteins concerned with production and secretion of antibodies from a cultured host cell. This enhancement is surprisingly synergistic, e.g., when combinations of targeted activators are directed to multiple locations along a promoter region of one or more transcription factors. For example, a cell line producing abundant antibody of interest can include CRISPR/dCas9 linked to a transcriptional activator, a first guide RNA (gRNA) having a spacer sequence complementary to a promoter region of a first transcription factor, and a second gRNA having a spacer sequence complementary to a promoter region of a second transcription factor. The first and second gRNAs are different from each other and the first and second transcription factors are different from each other. Further, the cells can include a third gRNA having a spacer sequence complementary to a promoter region of a third transcription factor different from the first and second transcription factors. The transcription factors are preferably those that increase expression of an antibody of interest.

In another aspect, transcription activator activity and production can be surprisingly and synergistically enhanced (e.g., greater than mere additive results) by directing two or more gRNAs to different locations of the same promoter. For example, many gRNA spacer sequences are about 20 bp in length, while many promoters regions range to about 200 bp or more. More than additive expression increases can be obtained, e.g., by directing three gRNAs with different spacer sequences to different promoter locations, e.g., toward the ends and center of the 200 bp region. Optionally, desirable improvements in expression can be obtained, e.g., using a single gRNA directed to the promoter for transcription factors PRDM1, XBP1 or IRF4, respectively.

In certain embodiments, the cell line is based on CHO, NS0, Sp2/0, or murine myeloma cell line X63Ag8.653. Often the cells are those previously expressing the desired antibody, but adjusted as described herein for enhanced expression. In alternate embodiments, the initial cells may be expressing a different antibody, only to be modified, as described herein, to produce a different antibody of interest.

The transcriptional activators of the expression enhancement system can be any functioning in relation to the protein product of interest. In many cases involving antibodies, the activators can be selected from one or more of VP64, VP16, and activation helper protein complex MS2-p65-HSF1 (MPH). For example, one or more different CRISPR/Cas9-Activator (and/or CRISPR/Cas12a-activator) complexes can be configured to increase expression of transcription factors such as those involved in expression of XBP1, IRF4, PRDM1, ELL2, SPI1, pTEFb, AFF4, AFF1, SUPT5HM, DOT1L, IRE1, PERK, BIP, SRP9, SRP14, and/or SRP54. Preferred transcription factors, particularly useful in enhancing expression of antibodies include, e.g., PRDM1, XBP1, and IRF4. The encoding nucleic acids can be endogenous to the host cell or recombinant. Transcription factors and/or their encoding nucleic acids can be endogenous to the host cells and/or incorporated by, e.g., transduction, transfection, electroporation, and/or the like.

In additional aspects of the inventions, the systems and cells can include alternate or complementary agents to provide enhanced expression. For example, the cells further include an inhibitor to expression of proteins possibly interfering with expression, such as AICDA, SIAH1, and HNRNP F. Optionally the host cells can be adapted to decrease binding of MLL2 protein to an IgH promoter or to an Eμ enhancer. In another option, the cells can be adapted to increase H3K4 methylation or increase H3K79 methylation at an IgH promoter or at an Eμ enhancer.

In a preferred embodiment the antibody production system includes one or more of the following options. A cell line producing an antibody of interest can have one or more cells with CRISPR/dCas9 (dCas9 disabled from cutting activity) linked to a transcriptional activator comprising VP64, the activation helper protein complex MPH, and/or the like. One or more first gRNAs have a spacer sequence complementary to the promoter region of PRDM1 or to a promoter region of a peptide downstream in a pathway from PRDM1. One or more second gRNAs have a spacer sequence complementary to promoter region of XBP1. And, one or more third gRNAs have a spacer sequence complementary to a transcriptional start site of IRF4. The cell line is, e.g., CHO, NS0, Sp2/0, Vero, HEK 293 or 293T and murine myeloma cell line X63Ag8.653, and the transcriptional activator is active in enhancing expression of the PRDM1, XBP1, or IRF4. The transcription factors function in increasing expression of the heavy and/or light chains in the antibody of interest. The cells can have the nucleic acid encoding the antibody present in the cell genome and/or in extra-genomic sequences.

The methods can enhance expression of an antibody of interest. For example, an expression method can include providing a cell with a nucleic acid with a sequence encoding a heavy chain of the antibody and a nucleic acid having a sequence encoding the light chain of the antibody. The cell is provided with CRISPR/Cas (e.g., CRISPR/Cas9 or CRISPR/Cas12) linked to a transcriptional activator as a complex. The complex is directed to a transcription factor encoding sequence by a first guide RNA (gRNA) spacer sequence complementary to the transcription factor promoter. The cell is provided with a second gRNA having a spacer sequence complementary to a promoter region of a second transcription factor. The first and second gRNAs are different from each other and the first and second transcription factors are different from each other. Expression of the transcription factors is promoted by the transcription activators attached to the Cas protein, and guided by the gRNA spacer sequences to increase expression of the heavy chain and/or light chain of antibody. The methods can experience exceptional benefits where one or more additional gRNAs are provided complementary to the first and/or second promoter, but at a different part of the promoter region. For example, CRISPR/dCas9-activator or gRNAs can be provided in the cells by any appropriate techniques such as, e.g., by electroporation, through Lentivirus transfection, or through encoded transposon sequences.

In a preferred embodiment, exceptional benefits in antibody production can be expected from the following conditions. It is preferred that the CRISPR/dCas9 be linked to a VP64, a VP16, or an activator helper complex MS2-P65-HSF1 (MPH) transcriptional activator. A powerful combination is presented when the promoter complementary to the first gRNA controls expression of PRDM1 or a peptide downstream in a pathway from PRDM1, the promoter complementary to the second gRNA is a promoter controlling expression of XBP1, and/or the cell is provided with a gRNA complementary to a promoter controlling expression of IRF4. In many embodiments, at least 3 of the gRNAs are used, or all three are used. The combination of features is useful in the context of host cell lines such as CHO, NS0, Sp2/0, and murine myeloma cell line X63Ag8.653.

The exceptional productivity of the engineered host cells of the invention can be passed on to production of alternate antibodies of interest, e.g., by swapping out antibody regions that provide specificity. For example, the methods can further include providing the expression host cell modified versions of the antibody heavy and/or light chain (or CDRs). The nucleic acid having the sequence encoding a heavy chain of the antibody and a nucleic acid having the sequence encoding the light chain of the antibody can be customized as follows. The cell is provided with CRISPR/Cas12 or CRISPR/Cas9 (Cas9 nuclease activity not deficient). A gRNA is provided with a spacer sequence complementary to a heavy chain sequence and/or to a heavy chain CDR target sequence endogenous to the cell. A gRNA is provided with a spacer sequence complementary to a light chain sequence or to a light chain CDR target sequence endogenous to the cell. An editing template nucleic acid is provided in the cell having a desired heavy chain sequence, light chain sequence, heavy chain CDR sequence, or light chain CDR sequence; the editing template also includes adjacent homologous regions complementary to sequences flanking the undesired target sequence (e.g., CDR3 of previously expressed antibody, no longer of interest) to be swapped out of the cell. The gRNAs are hybridized with their complementary target sequences. The targeted endogenous sequences are cut with the CRISPR/Cas at a site of gRNA hybridization. When the cut is repaired by homology directed repair (HDR) in the cell, the endogenous target sequence of the cell is replaced with the desired heavy chain sequence, light chain sequence, heavy chain CDR sequence, or light chain CDR sequence from the editing template. Thereby, the antibody expression of the cell is converted over from previous endogenous expression to expression of the antibody of interest.

The inventive expression systems include a gRNA multiplex expression system comprising a nucleic acid strand encoding two or more gRNA sequences each comprising a different spacer sequence to a different promoter region for the same transcription factor functionally associated with expression of an antibody. The transcription factor (TF) can be those appropriate to expression of antibodies, e.g., the TF consisting of XBP1, IRF4, PRDM1, ELL2, SPI1, pTEFb, AFF4, AFF1, SUPT5HM, DOT1L, IRE1, PERK, BIP, SRP9, SRP14, and/or SRP54.

The methods of enhancing expression of a target protein of interest include providing to a cell one or more nucleic acids encoding two or more gRNA sequences each comprising a different spacer sequence to a different region of a promoter for the same transcription factor promoter, and providing, e.g., CRISPR/dCas9 linked to a transcriptional activator to the cell. Such methods can result in target protein expression increased 4-fold or more over native endogenous expression levels (FIG. 4).

In one aspect, a gRNA multiplex expression system can comprise a nucleic acid strand encoding three or more gRNA sequences each comprising a different spacer sequence to a different region of a promoter for the same transcription factor. For antibody production, preferred transcription factor combinations include XBP1, IRF4, and PRDM1 or transcription factor for a protein downstream from PRDM1. For example, in such a system a first nucleic acid strand can encode three or more gRNA sequences each comprising a different spacer sequence to a different region of a promoter of the transcription factor PRDM1, and a second nucleic acid strand encodes three or more gRNA sequences each comprising a different spacer sequence to a different region of a promoter of the transcription factor IRF4.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification, the singular forms “a”, “an” and “the” can include plural referents unless the content clearly dictates otherwise. Thus, for example, reference to “a cell” can include a combination of two or more cells; reference to “bacteria” includes mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

As used herein, the term CRISPR refers to Clustered Regularly Interspaced Short Palindromic Repeats. CRISPR/Cas9 is a notoriously well-known complex of a Cas9 protein (having a nuclease activity) and a guide RNA (gRNA). The combination can target the nuclease activity to a precise location on a DNA strand. dCas9 (dead or disabled Cas9) is lacking in the nuclease activity, but retains the specific targeting ability in combination with a gRNA. CRISPR/Cas12a (Cpf1) is a complex of a Cas12 protein (having a nuclease activity) and a guide RNA.

gRNA, as used herein, is as commonly known in the art. The gRNA is a short RNA composed of a scaffold sequence necessary for Cas protein binding interaction and a spacer sequence (˜20 nucleotides) that defines a DNA target to be modified.

The term “promoter”, as used herein are as commonly known in the art. Promoter sequences are DNA sequences (typically 100 to 1000 base pairs) that promote binding of RNA polymerase, e.g., at a location upstream from the 5′ end of the transcription start site.

Transcription factors, as used herein are as commonly known in the art.

Transcription factors useful in the present methods and systems are typically polypeptides that bind to enhancer or promoter sequences to influence the rate of transcription of an associated gene. In many embodiments of the present inventions, CRISPR-dCas9 linked to a transcription activator is targeted to a promoter of a transcription factor known to stimulate expression of a gene involved in expression of an antibody. Particular transcription factors useful in the present cells, methods and systems are those involved in enhancing expression and/or secretion of antibodies. For example, such useful transcription factors include at least XBP1, IRF4, PRDM1, ELL2, SPI1, pTEFb, AFF4, AFF1, SUPT5HM, DOT1L, IRE1, PERK, BIP, SRP9, SRP14, and SRP54; transcription factors directed to proteins functionally downstream from PRDM1; and, more particularly, PRDM1, XBP1, and IRF4.

Transcriptional activators, as used herein, are activators, e.g., attached to the C-terminus of a Cas (e.g. dCas9 or Cas12a), to increase the expression of the transcription factors of the invention. The transcription activators can increase transcription of the transcription factors functioning to enhance the expression and/or secretion of an antibody of interest in a cell. For example, transcription activators commonly used in the present cells and methods can include VP64, VP16, and/or the activation helper protein complex MPH.

The term “endogenous”, as used herein, refers to those moieties native to a cell, as compared to exogenous moieties. For example, an endogenous gene is a gene originally in a host cell before it is modified by receipt of extraneous nucleic acids, e.g., by electroporation, genetic engineering, transfection, and/or the like.

### BRIEF DESCRIPTION OF THE DRAWINGS

FIG. 1A shows the generation of engineered fusion partner cell line containing master transcription factor regulatory elements by sequential development of the fusion partner cell line from the parental LA55 to FP21. Single cell clones derived from the parental LA55 cell line were clonally selected based on CD138 expression (FP19). FP19 cells were transfected with dCas9-VP64 and stable FP19-dCas9-VP64 expressing cells (FP19-dCAS9-complex) were selected for further development. Subsequently, MPH (MS2-p65-HSF1) was stably integrated into FP19-dCas9-VP64 cells to generate FP19c. FP20 and FP21 were generated by the stable integration of multiplex gRNA with and without S. aureus HLA-specific heavy and light chain genes (FP21 and FP20, respectively).

FIG. 1B shows the generation of an engineered CHO cell line demonstrating the origin and sequential development of the BREATH CHO cell line. Single cell clones derived from the CHO cell line were transduced with lentivirus containing dCas9-VP64 and MS2-p65-HSF1 and stably selected (CHO-dCas9 complex) for further development. Multiplex guide RNA (gRNA) was transduced in CHO-dCas9 complex cells and selected using Zeocin. The stable cell line was named as CHO-MTRE. Finally, CHO-MTRE were transfected with a mammalian expression vector expressing heavy and light chain genes of a monoclonal antibody against S. aureus HLA (AR-301) to generate the BREATH CHO cell line.

FIG. 2 show schematic diagrams of the Lentiviral constructs used for transduction of multiplex CRISPR of master transcription factor regulatory elements (MTREs) in a fusion partner cell line.

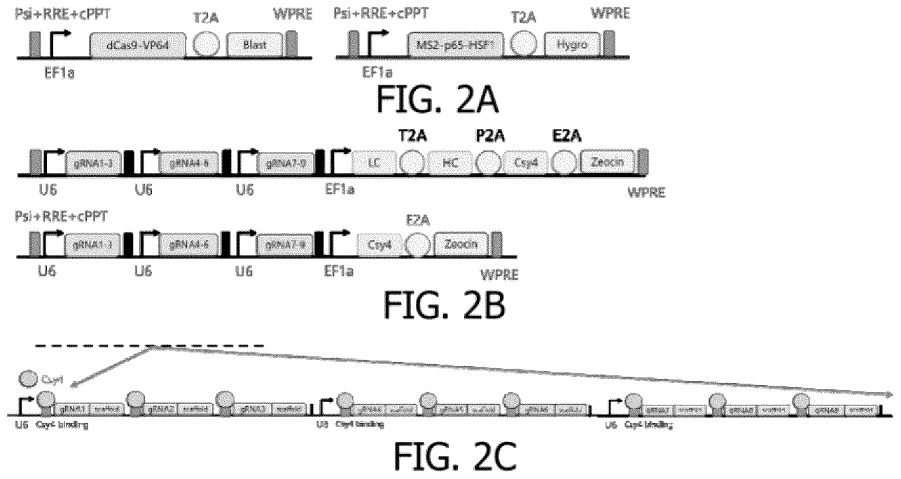

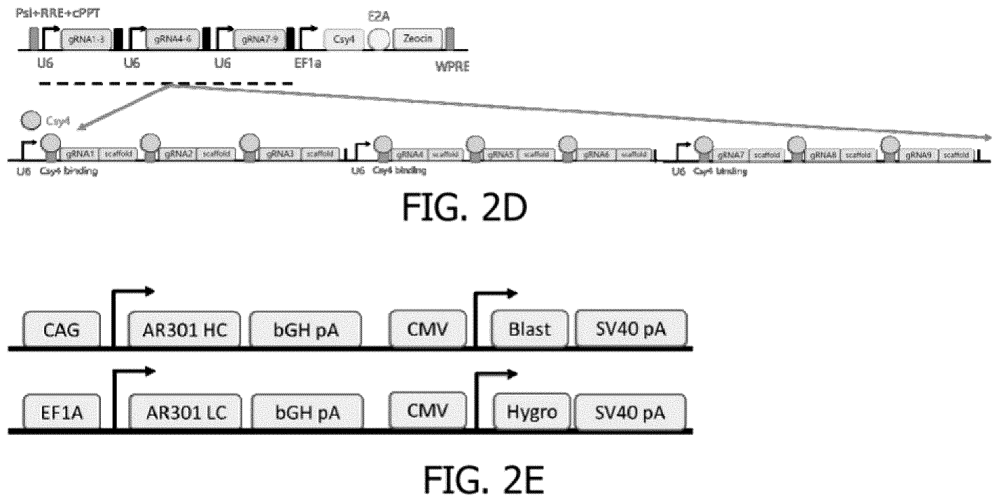

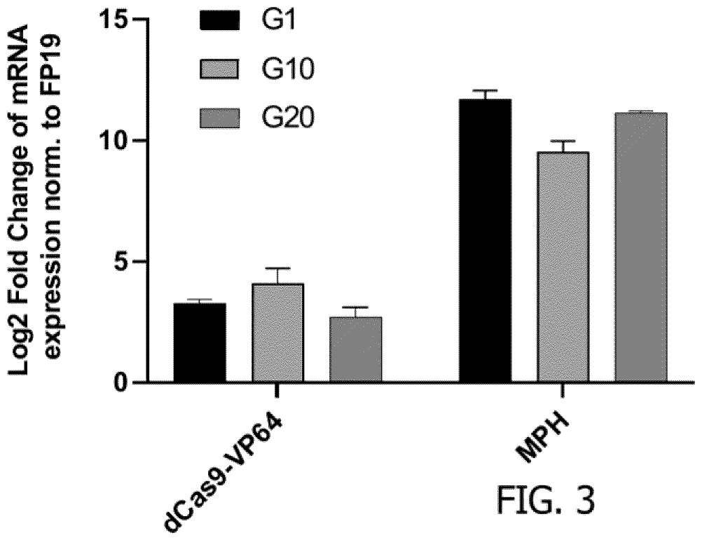

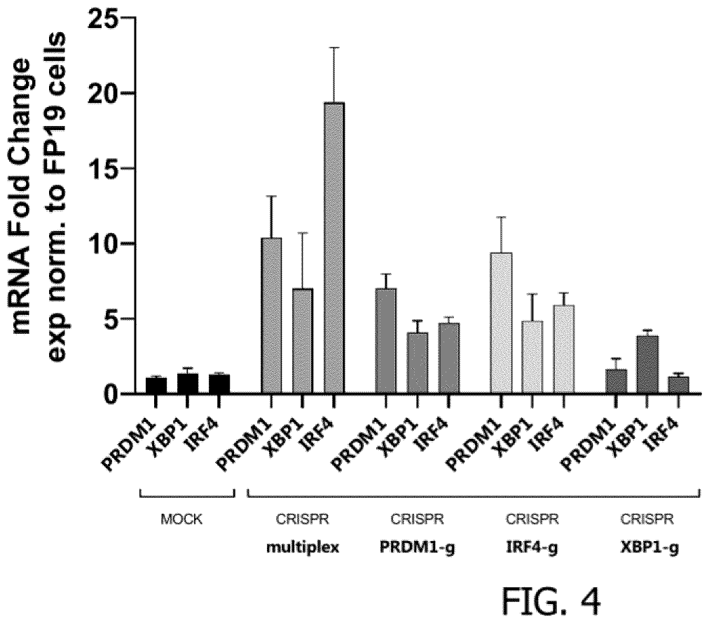

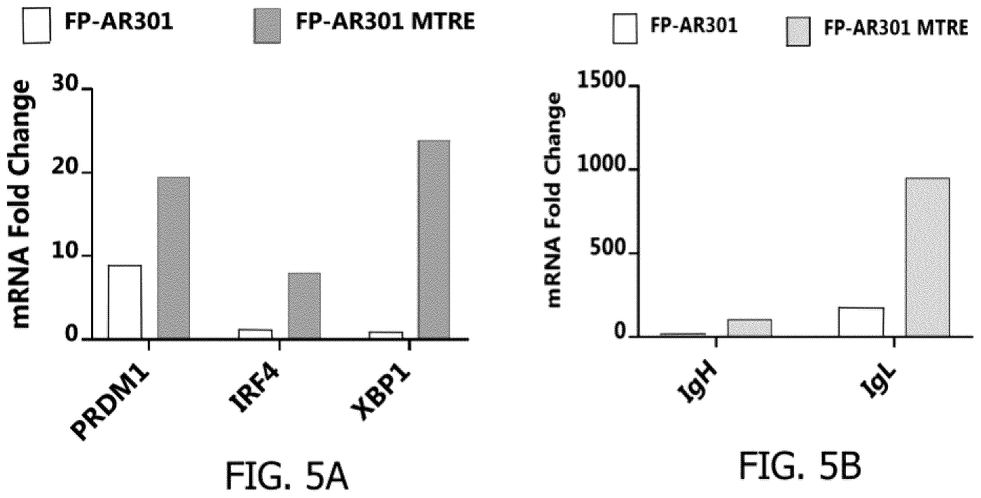

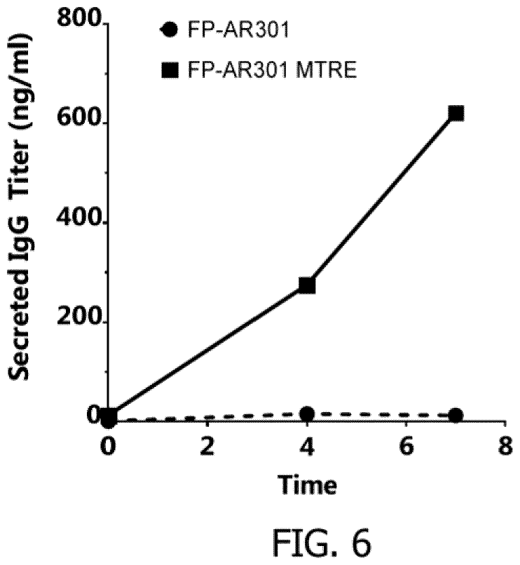

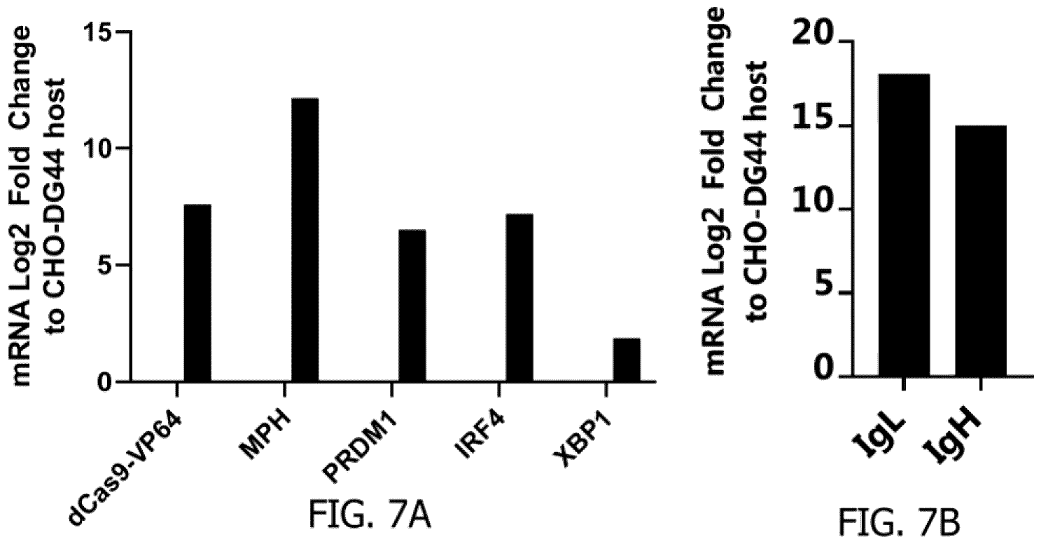

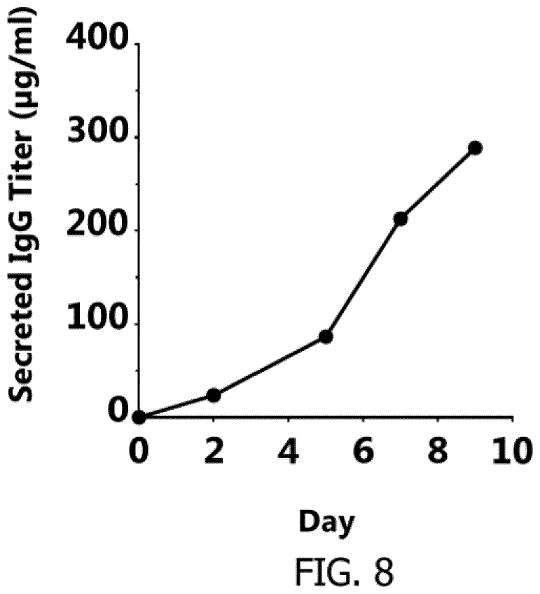

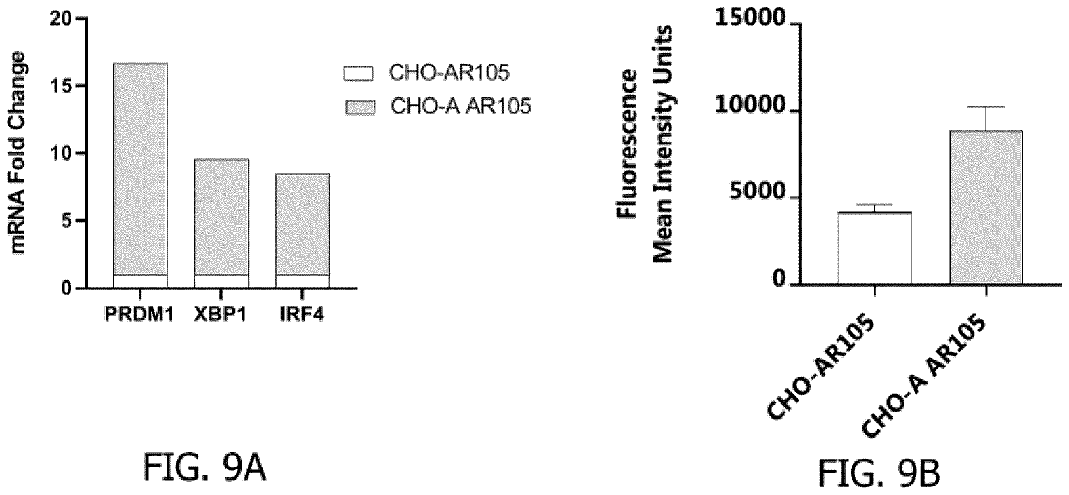

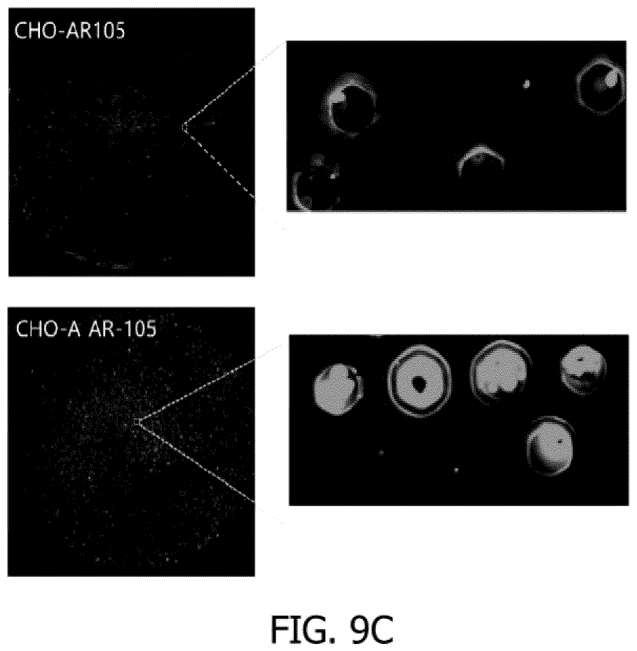

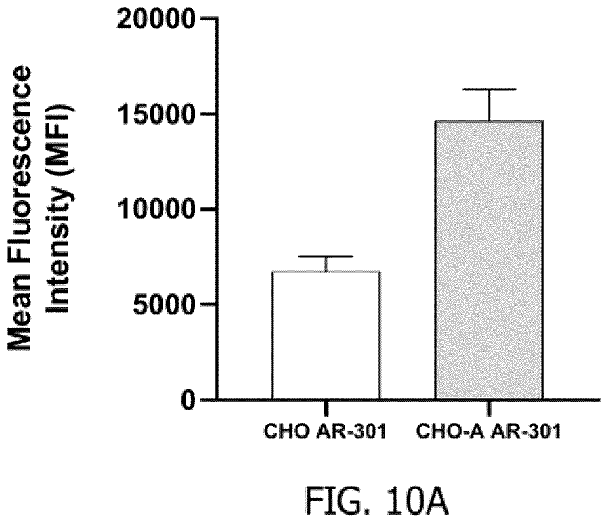

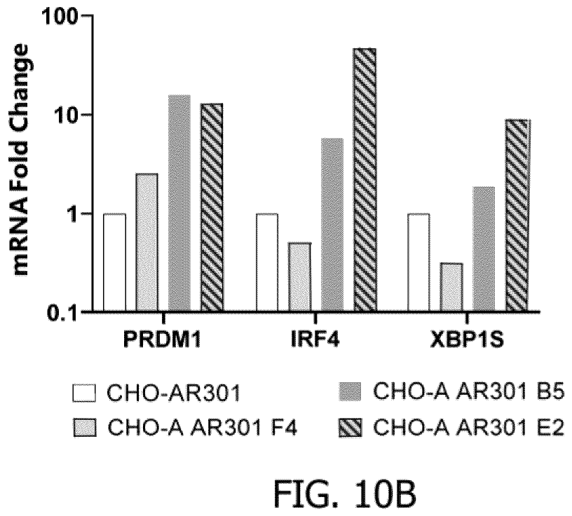

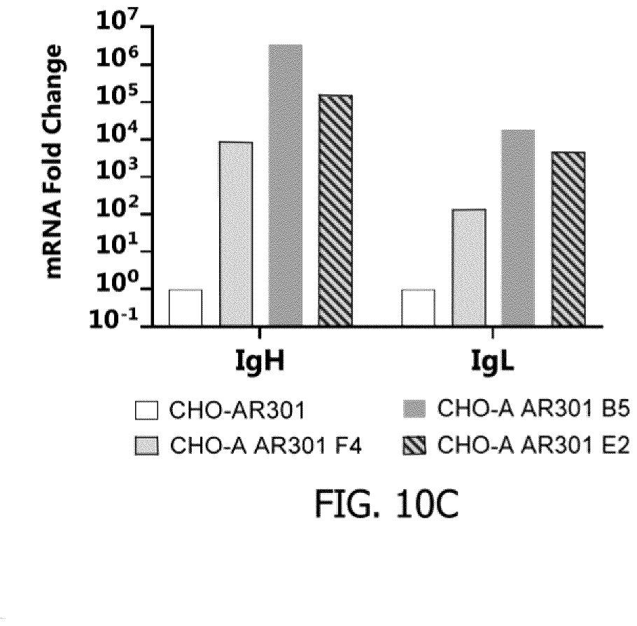

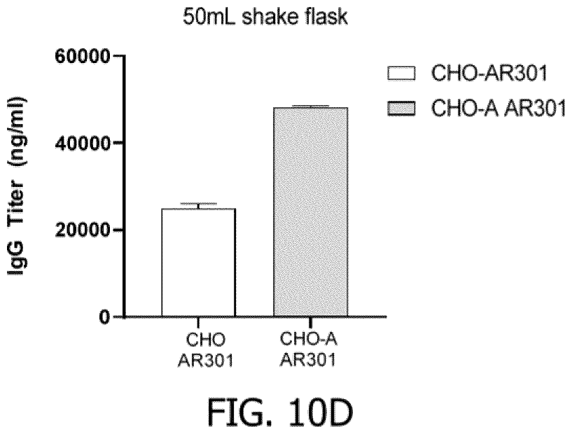

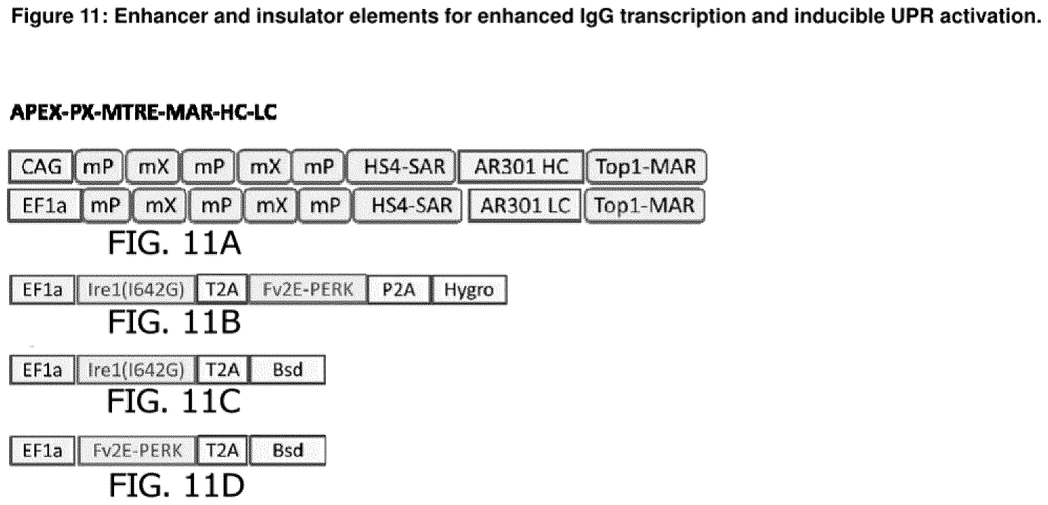

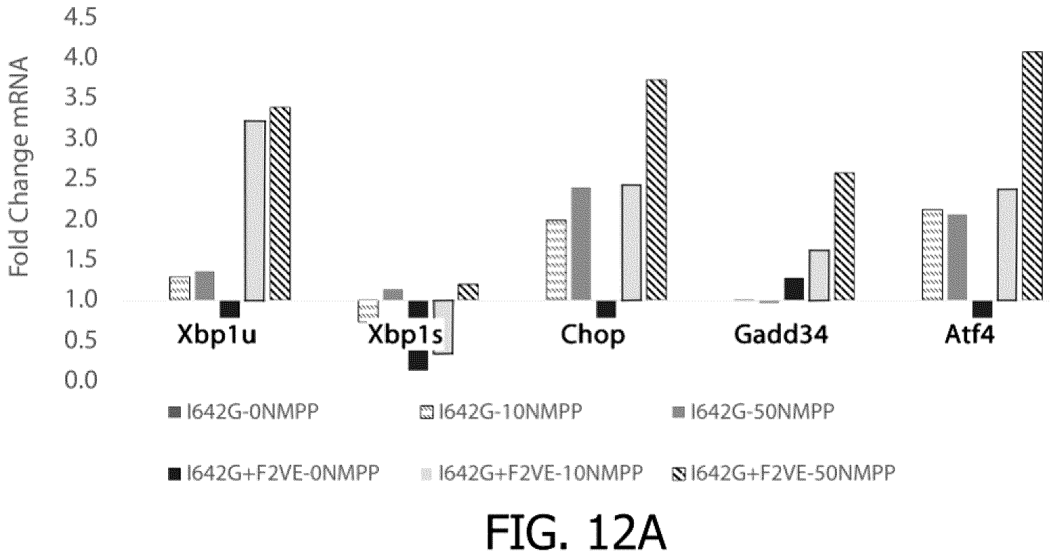

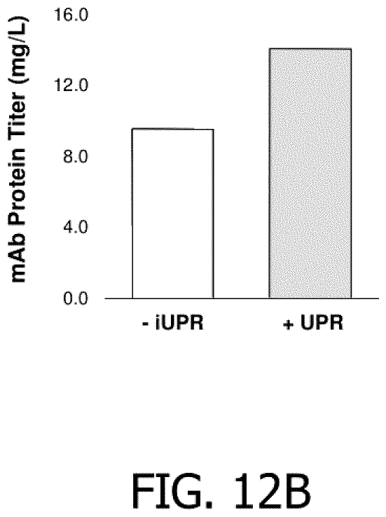

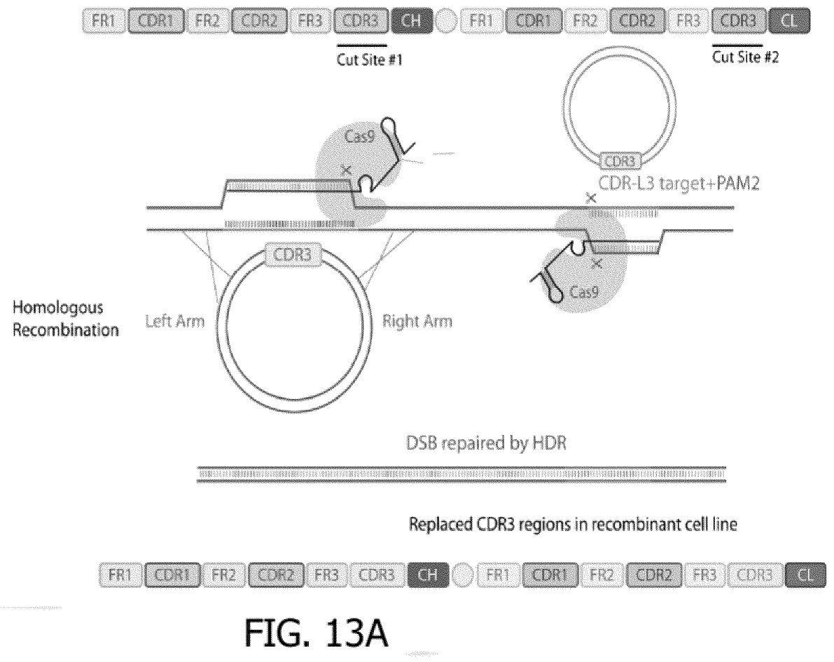

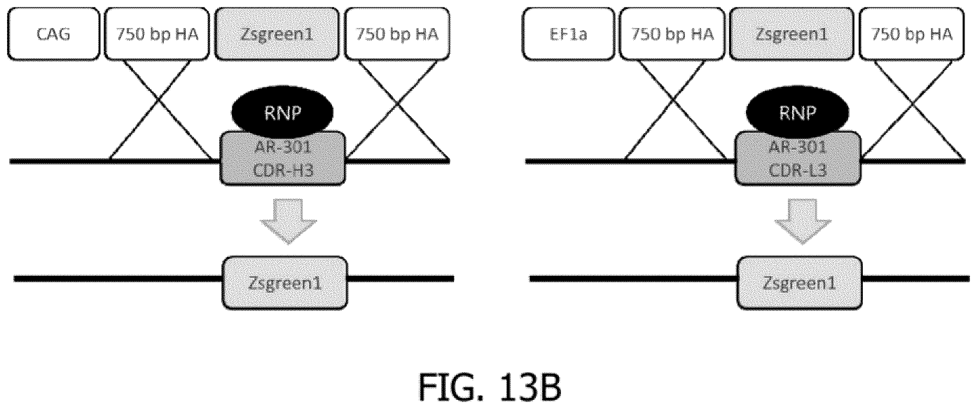

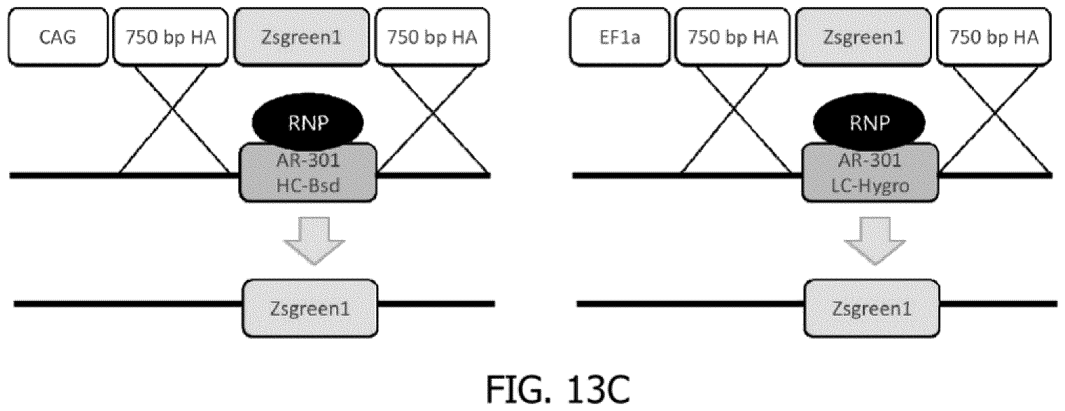

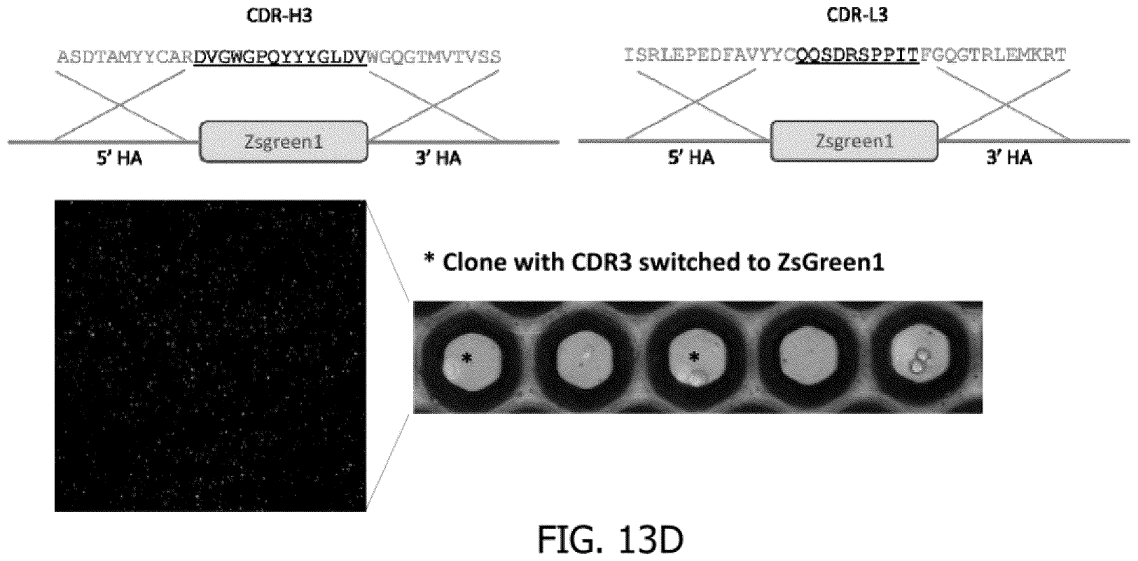

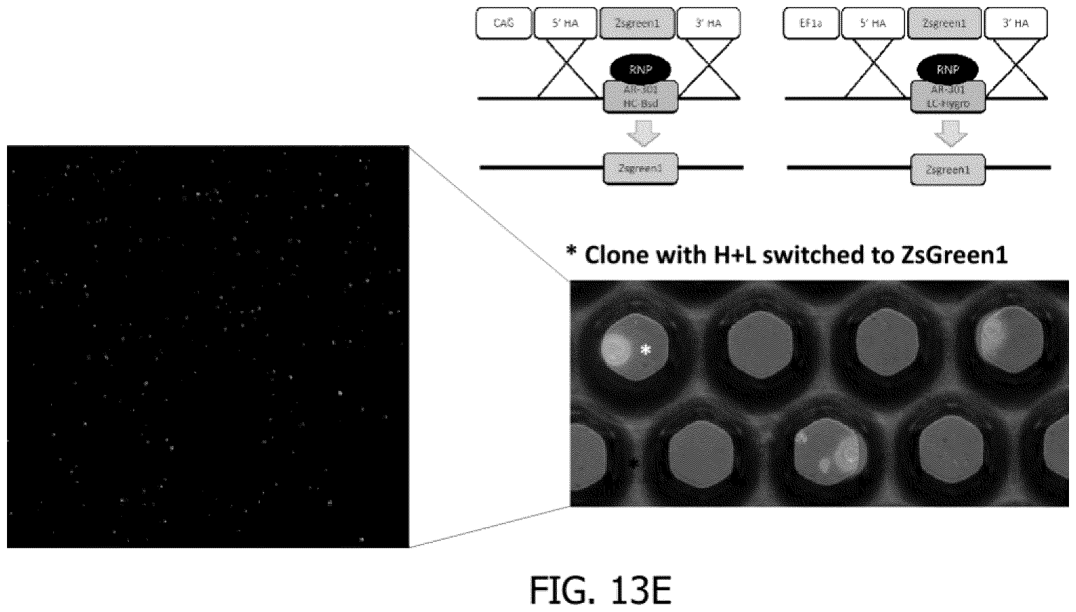

In FIG. 2A, lentiviral transfer vectors express dCas9-VP64 and Blasticidin (Blast) resistant gene (left) or accessory proteins, MPH (MS2-p65-HSF1) and Hygromycin (Hygro) resistant gene (right) are under the control of the EF1a promoter. Abbreviations: Psi+RRE+cPPT: psi signal, rev responsive element, central polypurine tract; T2A: Thosea asigna virus 2A peptide; WPRE: Woodchuck Hepatitis Virus (WHP) Posttranscriptional Regulatory Element; Self-cleaving peptides from Thosea asigna virus (T2A) is used for polycistronic expression.

In FIG. 2B, lentiviral transfer vectors containing a multiplex construct with (top) or without (bottom) human codon-optimized immunoglobulin heavy and light chain genes for expression in fusion partner cells. Self-cleaving peptides from Thosea asigna virus (T2A), porcine teschovirus-1 (P2A) and equine rhinitis A virus (E2A) are used for polycistronic expression. Zeocin is a Zeocin resistance gene for mammalian cell. Abbreviations: Psi+RRE+cPPT: psi signal, rev responsive element, central polypurine tract; U6: U6 promoter; HC Heavy chain; LC light chain; Cys4 Cys4 gene.

FIG. 2C shows an expanded depiction of the multiplex construct (dashed line in FIG. 2B) showing 3×3 modules to create a tandem of 9 gRNAs (SEQ ID NO: 6-14). The gRNA contains a 20 bp targeted region along with gRNA scaffold containing structure specific tetra-loop and stem loop modules and two MS2 binding sites.

In FIG. 2D, lentiviral transfer vectors are shown containing a multiplex construct in CHO cells. Bottom panel shows an expanded depiction of the multiplex construct (dashed line) showing 3×3 modules to create a tandem of 9 gRNAs. The gRNA contains a 20 bp targeted region along with gRNA scaffold containing structure specific tetra-loop and stem loop modules and two MS2 binding sites (SEQ ID NO: 15-25). In contrast to FIG. 2C, the multiplex construct in CHO cells does not have an expression cassette of heavy and light chain genes in the same vector.

FIG. 2E shows a schematic diagram of high-copy plasmid DNA vectors expressing AR301 HC (top panel) and AR301 LC (bottom panel) under strong ubiquitous promoters used for transfection and stable integration of heavy and light chain genes of AR301 monoclonal antibody (mAb) in CHO host cell line with MTRE activation. The two heavy and light chain cassettes are cloned in a high copy plasmid backbone, pUC19. Abbreviations: CAG hybrid promoter consisting of the cytomegalovirus (CMV) enhancer fused to the chicken beta-actin promoter; EF1A elongation factor 1 alpha; Blast Blasticidin resistance gene; Hygro Hygromycin resistance gene; SV40 pA poly A tail of simian virus 40; bGH pA poly A tail of bovine growth hormone. AR301 is an antibody binding HLA of S. aureus.

FIG. 3: Stable Expression of Transgenes in a FP19-dCas9 complex. CRISPR-mediated gene transduction of human-mouse heteromyeloma fusion partner (FP19) cells results in stable mRNA expression of transgenes dCas9-VP64 and MPH. Stable mRNA expression of transgenes over 60 cell generations are confirmed by the measurement of each transgene over time. FP19 cells were evaluated at generations 1, 10, and 20 (G1, G10, and G20) for mRNA expression of dCas9-VP64 and MPH (MS2-p65-HSF1) transgenes and confirmed to have similar levels of each mRNA transcript at each time point by RT-qPCR consistent with stable expression.

In FIG. 4, activation of single and multiplexed gene transcripts is demonstrated by fold change of mRNA level of each transcript in FP19 fusion partner cells before and after CRISPR mediated activation and in comparison to mock activation using scrambled gRNA. Cells containing stably integrated dCas9-VP64 and MS2-p65-HSF1 transgenes (FP19-dCas9 complex) were transduced with lentivirus containing all 9 nine gRNAs in a multiplex construct (multiplex), or single gRNA as labeled for each gene (PRDM1, IRF4, XBP1), or mock transduction (scrambled gRNA). The transcription levels for each gene were markedly increased when activated using the multiplex combination compared to single gene transduction consistent with synergistic gene activation. Transcript levels were measured by RT-qPCR for PRDM1, XBP1, and IRF4.

FIGS. 5A and B show enhanced transcription of MTRE, IgH and IgL genes in in fusion partner cells (FP-AR301 MTRE) via CRISPR-mediated MTRE activation measured by RT-qPCR. In FIG. 5A enhanced mRNA expression of PRDM1, IRF4 and XBP1 in FP21 (or FP-AR301 MTRE) is measured by RT-qPCR. Stable cell lines expressing AR-301 demonstrate up to 20 fold increase in MTRE mRNA expression (gray bars) in FP21 cells compared to baseline expression in non-activated cells for each transcriptional regulatory element (white bars). In FIG. 5B enhanced immunoglobulin mRNA expression of AR-301 in stable fusion partner cells (FP21, or FP-AR301 MTRE) is shown following MTRE activation measured by RT-qPCR. Stable cell lines expressing AR-301 demonstrate an increase in IgH and IgL mRNA expression with up to 1000-fold increase in IgL mRNA expression after MTRE activation (gray bars) in FP-AR301 MTRE cells compared to baseline expression in non-activated cells (white bars).

FIG. 6 shows enhanced secreted antibody productivity (secreted protein) in MTRE activated FP21 fusion partner cells measured by human immunoglobulin (IgG) sandwich ELISA. The chart is showing enhanced secreted antibody productivity following MTRE activation using features of the invention. Stable cell lines without (dashed line, FP AR-301) and with MTRE activation following the multiplex approach as disclosed (solid line, FP AR-301 MTRE) are shown for fusion partner cells expressing S. aureus HLA-specific mAb (AR-301). Antibody titers on days 0, day 4, and day 7 from cells in continuous culture were measured by human immunoglobulin (IgG) sandwich ELISA.

In FIG. 7A, CRISPR mediated activation of MTREs in CHO-DG44 (CHO MTRE) cell line demonstrates marked fold change in mRNA transcript levels as measured by RT-qPCR. Increased mRNA transcripts of dCas9 complex and MPH are shown. Increased mRNA transcripts of MTREs PRDM1, XBP1 and IRF4 by RT-qPCR are demonstrated. mRNA fold change expression relative to CHO DG44 host cell line without AR-301 antibody expressing genes was calculated based on 2^{-ΔΔCt}. mRNA transcript levels were normalized to GAPDH.

FIG. 7B demonstrates that CRISPR mediated activation of MTREs in CHO-DG44 host cell line (BREATH CHO) enhances mRNA transcript levels of IgH and IgL as measured by RT-qPCR. Transcript levels were normalized to ubiquitous housekeeping gene EIF3I.

FIG. 8 shows secreted AR-301 mAb protein production in CHO-DG44 host cell line containing activated MTRE expression. The increased mRNA expression of dCas9 complex, MPH, MTREs and IgH and IgL as shown by RT-qPCR in FIG. 7 is reflected in the increase in secreted immunoglobulin protein levels at shake flask scale. Shake flask expression of secreted protein production was measured by IgG sandwich ELISA for AR-301 mAb titer at different time points at Days 0, 2, 5, 7, and 9 of continuous production over 9 days. Cumulative antibody titer was measured.

FIG. 9 show enhanced antibody production in MTRE activated mAb-expressing CHO cell line, CHO-A AR105. AR 105 is a human monoclonal antibody, that binds to mucoid exopolysaccharide (MEP) of P. aeruginosa.

FIG. 9A shows increased PRDM1, XBP1 and IRF4 mRNA expression in CHO cell lines following MTRE activation. In FIG. 9A, a comparison of mRNA transcript levels is presented in non-activated (CHO-AR-105) and CRISPR multiplex activated CHO-AR105 (CHO-A AR105) cells demonstrating the marked increase in production of each mRNA transcript as measured by RT-qPCR following activation. mRNA transcript levels for PRDM1, XBP1 and IRF4 are shown for CHO-AR105 (without MTRE activation) and CHO-A AR-105 (with MTRE activation).

In FIG. 9B, CRISPR mediated multiplex activation results in a significantly increased production of IgG titer as measured by the increase in fluorescence mean signal intensity by single cells on nanowell arrays. The fluorescence intensity of each nanowell containing a single cell was measured 24 hours post-seeding of the CRISPR non-activated or activated CHO-AR105 production cell lines. Data represents the average fluorescence signal from approximately 500,000 cells per cell line (***p<0.0001).

FIG. 9C shows representative images of CHO AR105 and CHO-A AR105 clones, demonstrating clear visual differences in secreted antibody levels as measured by well fluorescence intensity with and without MTRE activation. Left panels show a population of 500,000 single cells clones of CHO AR105 on nanowells without (CHO AR105, top left panel) and with MTRE activation (bottom left panel, CHO-A AR105). Right panels show 8 individual nanowells showing fluorescent signal in individual clonal populations of CHO AR105 without MTRE activation (top right panel) and with MTRE activation (CHO-A AR105, bottom right panel).

In FIG. 10A, CRISPR mediated MTRE activation results in a significantly increased production of secreted IgG protein as measured by the increase in mean fluorescence signal intensity. Mean fluorescence intensity (MFI) is correlated to amount of secreted antibody deposited by cells along the walls of the nanowell arrays (Bobo et al., 2014). The fluorescence intensity (measured in mean intensity unit or MFI) of each nanowell containing a single cell was measured 24 hours post-seeding of the CRISPR non-activated (CHO-AR301) or activated CHO-A AR301 production cell lines. Data represents the average fluorescence signal from approximately 10,000 cells per cell line (****p<0.0001).

FIG. 10B shows increased PRDM1, XBP1, and IRF4 mRNA expression in CHO cell lines following MTRE activation measured by RT-qPCR. In FIG. 10B, a comparison of MTRE mRNA transcript levels is presented in non-activated (CHO AR-301) and CRISPR multiplex activated CHO AR-301 (CHO-A AR301) clonal cell lines demonstrating the marked increase in production of each mRNA transcript as measured by RT-qPCR following activation. mRNA transcript levels for PRDM1, XBP1 and IRF4 are shown for CHO AR-301 (without MTRE activation) and CHO-A AR-301 (with MTRE activation; clones F4, B5, and E2).

FIG. 10C shows increased immunoglobulin heavy chain (IgH) and light chain (IgL) mRNA transcripts in CHO cell lines following MTRE activation. mRNA transcripts of MTRE was measured by RT-qPCR. A comparison of mRNA transcript levels for IgH and IgL is presented in non-activated (CHO AR-301) and 3 different CRISPR multiplex activated CHO AR-301 (CHO-A AR301) cell lines (F4, B5 and E2) demonstrating the marked increase mRNA levels of each MTRE transcript.

FIG. 10D is a comparison of secreted protein (IgG titers) of MTRE activated (CHO-A AR301) and parental cell line (CHO_AR301). Following lead clone selection and single cell cloning on nanowells, CHO-A-AR301 were expanded and seeded for shake flask productivity (batch productivity). The cumulative endpoint IgG titer was measured at Day 7 post-seeding by human IgG sandwich ELISA.

FIG. 11 show schematic vector constructs for enhancer and insulator elements for enhanced IgG transcription (FIG. 11A; SEQ ID NO: 1-5) and inducible UPR activation (FIG. 11B-D).

FIG. 11A presents constructs for enhanced IgG expression. In the top panel, APEX-PX-MAR-HC-LC pseudoenhancer containing a chimeric HS4 insulator, scaffold/matrix attachment region (SAR) enhancers (HS4-SAR-Top1-MAR) is placed between CAG promoter and AR301 HC gene, and the EF1a promoter and AR301 LC gene respectively. Pseudoenhancer contains consensus binding sites for murine PRDM1 and murine XBP1 protein. To further stabilize gene expression, a Top1-MAR enhancer was placed at the 3′end of the heavy and light chain genes.

In FIGS. 11B to 11D, UPR activation constructs are shown. Three inducible constructs can be used to evaluate UPR activation for increased antibody production. Ire(1642G) allows for UPR activation through the use of an adenosine triphosphate (ATP) analog, 1NM-PP1. 1NM-PPI can attenuate Ire1 RNase activity via selective binding to the ATP pocket of the kinase domain of Ire1 (1642G) without affecting cell survival. Fv2E-PERK combined with treatment with AP20187 has a similar effect as Ire1 (1642G). In FIG. 11B, CHO Ire1(1642G) and Fv2E-PERK were combined together under the expression of EF1a promoter expressed bicistronically using T2A peptide. FIG. 11C and FIG. 11D are individual Ire(1642G) and Fv2E-PERK expressed individually under the control of EF1a promoter. Abbreviations: CAG, Chicken actin promoter and ß-globin enhancer elements; 2A self-cleaving peptides from Thosea asigna virus (T2A) and porcine teschovirus-1 (P2A); Hygro, gene to confer Hygromycin resistance; Bsd, gene to confer Blasticidin resistance.

In FIG. 12A drug-inducible activation of UPR pathway promotes ER expansion signaling pathways in a dosage-dependent manner. The Figure shows drug inducible UPR pathway activation in CHO-K1 cell line transfected with plasmid containing constitutive promoter expressing Ire1-1642G or Fv2E-PERK alone or in combination. At 24 hours post-transfection, cells were treated with varying concentrations of 1NM-PP1 (0, 10, or 50 μM). mRNA transcripts of UPR genes were measured by RT-qPCR of unspliced Xbp1 (Xbp1u), spliced Xbp1 (Xbp1s), Chop, Gadd34, and Atf4. Abbreviations: 1642G-0NMPP, IRE1(1642G) constitutive expression alone without 1NM-PP1; 1642G-10NMPP, IRE1(1642G) constitutive expression alone with 10 μM 1NM-PP1; 1642G-10NMPP, IRE1(1642G) constitutive expression alone with 50 μM 1NM-PP1; 1642G+F2VE-0NMPP, IRE1(1642G) and Fv2E-PERK constitutive expression together without 1NM-PP1, I642G+F2VE-10NMPP, IRE1(1642G) and Fv2E-PERK constitutive expression together with 10 μM 1NM-PP1; 1642G+F2VE-50NMPP, IRE1(1642G) and Fv2E-PERK constitutive expression together with 50 μM 1NM-PP1.

In FIG. 12B, drug-inducible activation of UPR pathway enhances production of secreted AR-301 immunoglobulin protein levels. The figure shows drug inducible UPR pathway activation in stable cell line expressing AR-301 mAb by co-transfection of plasmids containing constitutive promoter expressing Ire1-1642G and Fv2E-PERK (+UPR) or mock (−UPR; FIG. 12B). At 24 hours post-transfection, cells were treated with 10 μM of 1NM-PP1. After 24 hours post-treatment of 1NM-PP1, CHO-AR301 cells with and without UPR activation were seeded at the same cell density (3E5 viable cells/ml) in 20 ml seeding volume for shake flask production run for 7 days. Secreted IgG protein levels of AR-301 mAb at Day 7 with and without UPR activation were measured by IgG sandwich ELISA.

FIGS. 13A to 13E demonstrate a method of editing of CHO host cell line CDR domains to change the antibody specificity from one mAb to a different mAb. FIG. 13A shows the use of CRISPR directed homologous recombination to enable site specific integration of CDR3s into the engineered host cell lines. Specific CDR3 targeting and replacement is achieved using the Protospacer Adjacent Motif (PAM) and gRNAs that are designed to precisely target the CDR3 sites. Switching of CDR3 domains was performed by introducing single stranded donor DNA containing the new CDR3 by the use of adeno-associated virus 2 (AAV2). The same approach was used for swapping out heavy and light chain genes. DSB: double stranded break

FIG. 13B presents switching of CDR3 Heavy and Light chain genes into CHO Host Cell Lines by CRISPR directed homologous recombination. The schematic demonstrates a method of replacement of complementarity determining region 3 (CDR3) for heavy and light chain genes of BREATH CHO host cell line with transgene such as Zsgreen1 fluorescent protein. A ribonucleoprotein (RNP) complex consisting of Cas9 and sgRNA was assembled in vitro. The sgRNA targets 5′ and 3′ end of AR-301 CDR-H3 and CDR-L3 (SEQ ID NO: 36-37, SEQ ID NO.: 48-49. Following RNP entry into the cell by electroporation, editing occurs via homology directed recombination (HDR). Edited CHO cells are selected for the presence of ZsGreen1 fluorescence and absence of IgG secretion.

In FIG. 13C, IgH and IgL Genes are switched into CHO Host Cell Lines by CRISPR directed homologous recombination. The schematic demonstrates the CHO host cell line containing stably integrated IgH and IgL (HC and LC) genes mAb, AR-301 HC-Bsd, and AR-301-LC-Hygro respectively and method of replacement of the IgH and IgL with an exogenous transgene such as Zsgreen1. Ribonucleoprotein (RNP) complex consisting of Cas9 and sgRNA are assembled in vitro. The sgRNA targets the 5′ and 3′ end of the AR-301 HC and LC (SEQ ID NO: 26-35, SEQ ID NO: 38-47). Following RNP entry into the cell by electroporation, editing occurs via homology directed recombination (HDR). Edited CHO cells are selected for the presence of ZsGreen1 fluorescence and absence of IgG secretion.

In FIG. 13D, CDR-H3 and CDR-L3 are switched into CHO Host Cell Lines by CRISPR directed homologous recombination. The top panel shows the schematic for swapping of the Zsgreen1 cassette (containing stop codon, TGA, followed by Zsgreen1 cassette containing CMV promoter, coding region of Zsgreen1 fluorescent protein and SV40 polyA tail). The cell with CDR3 switched to Zsgreen1 cassette is shown by the presence of green fluorescence signal but no secreted antibody signal (measured by red fluorescence, red). Detection of a successful gene swap was performed at 72h following RNP delivery. The edited CHO cells were seeded in nanowells coated with anti-human IgG capture antibody. Secondary detection antibody conjugated to Cy3 was added to detect for clones secreting the parental full-length heavy and light chain antibody genes of AR-301. Images were acquired at 24 hours post-seeding. The bottom left panel shows 60,000 nanowells while the bottom right panel is a magnified inset showing successful switching of CDR-H3 and CDR-L3 to Zsgreen1 cassette.

In FIG. 13E, heavy and light chains in CHO Host Cell Lines are switched by CRISPR directed homologous recombination. The top panel shows the schematic for swapping of Zsgreen1 cassette (containing only coding region of Zsgreen1 fluorescent protein and SV40 polyA tail). Cells with IgH (heavy chain) and IgL (light chain) switched to Zsgreen1 cassette are detected by the presence of green fluorescence signal but no secreted antibody signal (measured by red fluorescence, red). Detection of successful gene swap was performed at 72h following RNP delivery. The edited CHO cells were seeded in nanowells coated with anti-human IgG capture antibody. Secondary detection antibody conjugated to Cy3 was added to detect for clones secreting the parental full-length heavy and light chain antibody genes of AR-301. Images were acquired at 24 hours post-seeding. The bottom left panel shows 60,000 nanowells while the bottom right panel is an inset showing successful switching of heavy and light chain genes to Zsgreen1 cassette.

### DETAILED DESCRIPTION

The invention refers to, e.g., a modular and customizable multiplex CRISPR/Cas system for the development of clonal cells lines for high level production of monoclonal antibodies with demonstrated stability, and high level productivity with reduced process development timelines. The system is based on optimization of cell lines by leveraging nascent enhancers and chromatin states combined with site specific gene editing with precise molecular control. Also, described are novel systems for changing the target of an antibody expressed in a host cell. Further, the combination of expression enhancement and antibody target switching technologies can drastically expedite production and productivity of new antibodies.

The present inventions include cells, cell lines, and systems producing exceptional quantities of antibodies, e.g., compared to current typical antibody expression systems. For example, novel combinations of synergistically complementary elements are described to drastically increase antibody production in their modification of, e.g., standard traditional expression cell lines. In many embodiments, a transcription activator attached to a CRISPR/dCas9 or CRISPR/Cas12a is directed to a promoter region near the transcription factor transcription start site (TSS) by a guide RNA (gRNA). The transcription factor is associated with antibody expression and/or secretion. Thereby, the direction of the CRISPR/Cas transcription activator to the transcription factor TSS by the gRNA results in over production of the transcription factor associated with antibody production. By directing transcription activators to several different transcription factor promoter regions, antibody production can be enhanced even more. Further, as a surprisingly synergistic result, better than additive expression gains can be provided by gRNA direction of the transcription activators to multiple locations on the same promoter.

In a complementary technology, site-directed CRISPR/Cas homology directed repair (HDR) can be used to rapidly convert a host cell efficient in production of one antibody to produce a different desired antibody. This can greatly speed up production and validation of the different antibody.

The present methods employ the cells and systems described herein. For example, starting with an expression host cell already expressing an antibody, elements are emplaced to accelerate expression and/or secretion of the antibody. The necessary elements can be introduced into the host cell by, e.g., electroporation. Introduced gRNAs can direct CRISPR/Cas (typically employing dCas9 or Cas 12a) to target promoter regions associated with transcription factors known to influence control over antibody production. Additional benefits are available by targeting promotor regions of multiple transcription factors and by targeting multiple locations on the individual transcription factor promotor regions.

A number of methods and compositions are discussed in the Summary of the invention and further details are provided herein and in the Examples section. As would be readily appreciated by the skilled person, the disclosures can be read in combination.

A Method to Increase Antibody Production by activation of endogenous master transcription regulatory elements (MTRE) including but not limited to PRDM1, XBP1, and IRF4.

To engineer a genetically stable cell that produces high titer mAb, we first looked to the native machinery of antibody production in plasma cells. The antibody-secreting cell (ASC) compartment has been very well studied in murine models consisting of short-lived proliferating plasmablasts (PBs) and long-lived post-mitotic plasma cells (PCs).

Background: An extremely high rate of immunoglobulin secretion in plasma cells (up to 300 pg/cell/day) is achieved with highly specialized morphology with enlarged cytoplasm and tightly arranged endoplasmic reticulum (ER). Plasma cells (PC), the major antibody-secreting cells, are derived from B lymphoblasts and are the final effectors of the B-cell lineage. The transcriptional mechanisms that are required for function of activated B cells and plasma cells are different and appear mutually exclusive. In activated B cells, BCL-6 (B-cell lymphoma 6), MTA3 (metastasis-associated 1 family, member 3), PAX5 (paired box protein 5), and MITF (microphthalmia-associated transcription factor) are important for inducing B-cell gene-expression programs, but also repress plasma-cell formation. PCs also constitutively activate the unfolded protein response (UPR), a specialized sensing mechanism to detect and deal with large amounts of protein passing through the ER (Tellier, 2016). The defined developmental program of activated B cells into PCs requires a triad of PC-specific genes: IRF4, BLIMP-1 (encoded by PRDM1) and XBP1. Such a developmental program requires highly coordinated activation and silencing of hundreds of targets genes, including B-cell fate factors. In primary human B cells, deletion of these genes results in drastic decrease of antibody secretion level. In murine, this triad is required and sufficient for antibody secretion at the germinal level (Shapiro-Shelef, M., et al. 2005).

PRDM1 and IRF4 regulate the transcription of IgH genes via the enhancer elements HS1, HS2, and HS4 and indirectly decrease the level of E3 ubiquitin ligase, Siah1, a key factor for degradation of immunoglobulin via the ERAD pathway (Tellier 2016; Swaminathan 2015). It is also important to note, as explained previously, MAR enhancer elements function to enhance the native Eμ enhancer of plasma cells, which is key in regulating Igh/Igl transcription.

PRDM1 and XBP1 are required for immunoglobulin secretion by plasma cells. PRDM1 regulates the μM (transmembrane form of u immunoglobulin heavy chain) to μS (secreted form) mRNA switch. By repression of PAX5, it derepresses transcription of the genes that encode the immunoglobulin heavy chain, the immunoglobulin light chain, and XBP1, which are required for organelle biogenesis, endoplasmic-reticulum function, and protein folding and protein secretion.

In addition to PRDM1 and IRF4, IGH gene transcription and post-transcriptional processing is regulated by elongation factor ELL2 in plasma cells. IGH transcription promotes the loading of polyadenylation factors CSTF and CPSF onto phosphorylated RNA Polymerase II (RNAP-II) at Ser-2 on the carboxyl-terminal domain. ELL2 transcription is activated by PRDM1 and IRF4 (Sciamma 2004; Shaffer 2004). ELL2 promotes secreted IGH via polyadenylation factor CSTF-64 (Martincic 2009) whereas depletion of ELL2 or its upstream activator, heterogenous ribonucleoprotein F (HNRNP F) decreased secretory-specific forms of immunoglobulin heavy-chain mRNA. At the IGH promoter, ELL2 induces methylation of H3K79 and H3K4, binding of DOT1L, and decreases the levels of MLL bound at the IGH promoter as well as E3 ubiquitin ligase SIAH1. SIAH1 can promote posttranslational degradation of unfolded immunoglobulins via the ERAD pathway (Swaminathan 2015).

Within the IGH locus, the Eμ intronic enhancer controls IGH transcription. The Eμ consists of small 220 bp core element two flanking nuclear matrix attachment regions (MAR). MAR have been demonstrated to further enhance Eμ by promoting transcription. The regulation of IGL transcription is regulated by PU.1.

In plasma cells, the endoplasmic reticulum (ER) is a critical organelle for immunoglobulin protein synthesis, folding and modification. Dysregulation of ER functions leads to the accumulation of misfolded- or unfolded-protein in the ER lumen, and this triggers the unfolded protein response (UPR), which restores ER homeostasis essential for normal immunoglobulin synthesis and secretion in antibody-secreting plasma cells.

XBP1 also plays an indispensable role in the endoplasmic reticulum (ER) expansion in plasma cells. XBP1 upregulates the expression of UFBP1 and UFMylation pathway genes in plasma cells, while UFBP1 deficiency impairs ER expansion in plasma cells and retards immunoglobulin production. Structure and function analysis indicate that UFBP1 is required for immunoglobulin production and stimulation of ER expansion in IRE1α-deficient plasmablasts and regulates different branches of the UPR pathway to promote plasma cell development and function (Zhu, H. et al, 2019).

The present invention leverages the core components of the plasma cell machinery to further enhance antibody secretion in production cell lines. For example, productivity benefits can be realized by (1) increasing PRDM1, IRF4, and XBP1 transcription; (2) enhancing IGH and IGL transcription; and/or (3) activation of secretory and unfolded protein response (UPR) to promote antibody biosynthesis.

CRISPR/Cas technology. Methods for genetic engineering and control of gene expression have utilized naturally occurring transcription factors (TFs) and manipulation of regulatory elements of targeted genes. Although engineered nucleases such as transcription activator-like effector nucleases (TALENs), zinc-finger nucleases (ZFNs) have been used, the complexity of their synthesis and design criteria have limited their use. These challenges have been addressed using RNA-guided clustered regularly interspaced short palindromic repeats (CRISPR) combined with Cas endonuclease enzymes, such as (CRISPR-Cas9 and CRISPR/Cas 12a). The functions of the CRISPR Cas enzymes has expanded beyond gene editing. Cas9 mainstream applications include generation of deletions and insertions via induction of a site-specific double strand breaks (DSB) followed by repair by either Homology Directed Repair (HDR) or Non-homologous End Joining (NHEJ). The Cas9 toolkit has further been expanded to gene activation (CRISPRa) and gene repression (CRISPRi) (Cho et al., 2018). With regard to CRISPR/Cas 12a, similar functions are enabled, but, e.g., with typically more precise targeting to target DNA, e.g., due to the staggered target cutting.

Several variants of Cas9 currently exist and include (1) wild type Cas9; (2) HDR-specific Cas9 (D10A mutations); and (3) dead Cas9. The dead Cas9 (dCas9) contains mutations in HNH and RuvC domains (H850A, D10A) which abrogates its nuclease activity. This variant, when fused with various effector domains (transactivation, repressor, or chromatin remodeling proteins) can either enhance or repress transcription.

In order to achieve precise CRISPR-Cas mediated gene editing, e.g., a 20-nucleotide target sequence of a single guide RNA (gRNA) is required. The sequence specificity provides a specific target and cleavage site for the CRISPR-Cas9 gene editing complex. This complex can consist of CRISPR RNAs (crRNAs), fused to a trans-activating domain (tracrRNA), a 2-5 nucleotide consensus sequence, species-specific protospacer-associated motif (PAM) following immediately 3′ of crRNA complementary sequence (e.g. target DNA). The ability to target specific domains as a DNA binding protein enables Cas9 to recruit repressor or activator domains resulting in modifications to transcription.

We describe herein an approach for transcriptional activation that goes beyond previously described technology with surprisingly synergistic results. For example, the present invention allows for simultaneous binding and processing of 9 unique gRNAs by utilizing the Csy4 endoribonuclease that processes RNA in a site-specific manner. Csy4 cleaves the RNA through a 28-nt hairpin sequence at the 3′ end of the stem. The unique structural modality allows for simultaneous transcriptional activation as opposed to repression.

gRNAs/RNA Scaffold

The engineering to generate an appropriate gRNA scaffold is application-dependent. The structure of the gRNA as well as the delivery method used for transcriptional activation is markedly different compared to that required for editing where the sgRNA scaffold contains tracrRNA.

Multiplex Processing of gRNA Using Csy4

The Multiplex CRISPR technology approach utilizes a unique and modular gRNA scaffold design for multiple processing of gRNAs in a single vector containing up to 9 gRNAs. This has facilitated the synergistic activation of multiple genes in the sample molecular pathway superior to a single gRNA activation, e.g., as shown in FIG. 4.

Our method of CRISPR activation (CRISPRa) has employed, e.g., three gRNAs driven by a single U6 promoter and contains 28 nt hairpin sequence (CSY4 binding), a tetraloop, and two stem-loop structures. In addition, an MS2 aptamer binding site, one attached at stem loop 2 and one attached at the tetraloop was used to further enhance transcriptional activation. Finally, an RNA Pol III terminator sequence is included following every third gRNA (FIG. 2).

Key transcription factors: The features of our invention utilized key plasma cell transcription factors associated with antibody production in primary human plasma cells to increase antibody production in cell lines not of B cell origin. Increased immunoglobulin transcription and protein synthesis is associated with upregulation of master transcription factors PRDM1, IRF4, and XBP1 which were subsequently selected.

Promoters used: EF1alpha promoters were selected to drive the expression of dCas9-VP64, MS2-p65-HSF1 to work in tandem to amplify antibody production. Promoter activation is universal and can be applied to any host cell irrespective of B cell lineage.

Lead 20 bp target MTREs are selected based on the distance to their respective transcription start sequence (TSS). It has been demonstrated that this optimal distance is within 200 bp of the TSS (Konermann et al., 2015). We elected to use a nuclease-dead Cas9 for Streptococcus pyogenes dCas9 (D10A/H840A) fused to transactivation domain of VP64. Additionally, we generated a fusion protein similar to Konermann et al., 2015 using a heteroeffector fusion protein: MS2-p65-HSF1. p65 recruits AP-1, ATF/CREB, and SP117, whereas VP64 recruits PC418, CBP/p300, and the SWI/SNF complex. Addition of HSF1 to the triplex results in synergistic transcriptional activation for hundreds of targets (Konermann et al., 2015). We designed the sgRNA scaffold with tetraloop and stem-loop 2 and two MS2 binding sites.

In a preferred embodiment, the invention refers to a specific application of CRISPR-Cas9 gene activation structure for the activation of mAb H&L genes in production cell lines by activating PRDM1, XBP1, IRF4 transcription factors (TF) simultaneously (multiplex activation) to increase antibody production of cell lines such as CHO and myeloma fusion partner cell lines. The present invention includes the following key features for antibody enhancement:
A modular system in which activated promoters are designed to work in tandem to amplify antibody production; 3 U6-promoters (Pol III) are placed in tandem and each U6 promoter can drive expression of three distinct gRNAs.
3 specific gRNAs are designed for each TFs being targeted which flank the 5′, middle, and 3′ regions of the transcription start site (TSS; SEQ ID 6-25).
Dual promoter system: stable expression of gRNA triplex array by U6 promoter and EF1a promoter to drive stable expression of Csy4 and LC/HC (see FIG. 11C); modified origin of replication for high copy amplification of plasmid DNA.
Promoter activation is universal and can be applied to any host cell irrespective of B cell lineage.
The order of transduction of the components (Cas-9, accessory proteins, gRNA) into the mAb production cell using a lentivirus vector system.

### General Methods

Lentivirus packaging and titer. Lentivirus was packaged using Lenti-X Packaging Single Shots (VSV-G) and 293FT containing large SV40 antigen (Invitrogen). 293FT was grown in media recommended per manufacturer's instructions. 1 day prior to transfection, 293FT were re-seeded to achieve 70-80% confluency in T175 flask the next day without the use of antibiotics. 20 μg of DNA of lentivirus transfer plasmids were added into 293FT with Lenti-X Packaging Single Shots (VSV-G) in a 600 μl reaction mixture. Virus was harvested either at 48- or 72-hours post-transfection and titer was determined by p24 ELISA (Takara) by standard protocols.

7-day kill curves for engineered fusion partner and CHO cell lines. A 7-day kill curve for FP19 was performed using culture conditions with Blasticidin (Blast), Hygromycin (Hygro), Zeocin (Zeo) and puromycin (Puro) as single agents. 20,000 cells/well seeded on Day 0 in 0.4 ml of Hybridoma-SFM media. Cell counts were performed at Days 0, 4, and 7. The concentration of each antibiotic used for selection was determined by the lowest kill concentration. Double antibiotic combinations of blast+hygro and triple selection of Blast+Hygro+Zeocin were also determined. For determination of antibiotic selection concentration for CHO DG44-deroved cell lines, 20,000 cells/well seeded on Day 0 in 0.4 ml of EX-Cell CD CHO Fed-Batch in 6 mM L-Glutamine, 1% hypoxanthine and thymidine (HT). Cell counts were performed at days 0, 4, and 7. Antibiotic selection concentration using Blasticidin and Hygromycin were determined by the lowest kill concentration.

Transduction of engineered cells derived from FP19 and CHO. FP19-derived cells (1×10⁶) were transduced at a MOI of 5 and 10 with the addition of 8 μg/ml Polybrene to improve transduction efficiency. At 48 hours after transduction, antibiotic selection was added at 0.1× of the lower kill concentration from 7-day kill curves. Antibiotic selection was slowly increased to a final concentration of 20 μg/ml over the course of 4 weeks for stable selection. CHO DG44-derived cells (1×10⁶) were transduced at an MOI of 10 in the presence of 8 μg/ml of polybrene.

Transfection of plasmid DNA of CHO-derived cells. 20 μg of plasmid DNA containing AR301 HC and AR301 LC were transfected into 1×10⁷ cells per reaction using Mirus Trans-IT Pro transfection reagent by following manufacturer's guidelines (Mirus Bio).

Vector construction of constructs with multiplex Cas9-directed MTRE activation: Master lentiviral transfer vectors were generated by modifications to pLenti6.2/V5-DEST Gateway (Reference) where CMV promoter and enhancers, chloramphenicol, attP docking sites, and V5 tag were removed. The vector was re-engineered to include a high-copy origin of replication, multiple cloning sites, EF1alpha promoter driving antibiotic resistance markers Blasticidin, Zeomycin, Hygromycin or puromycin. All vector elements were reassembled by Gibson assembly.

Vector construction of pLV-EF1alpha-dCas9-VP64-Blast and pLV-EF1alpha-MS2-p65-HSF1 (MPH)-Hygro: Human codon-optimized dCas9-VP64 and MS2-p65-HSF1 were synthesized de novo containing BsiWI/EcoRI cloning sites and cloned in frame to T2A peptide and Blasticidin or Hygromycin resistant gene (FIG. 2A).

A multiplex gRNA vector that contains U6 promoter, 28-nt Csy4 binding site, BbsI cloning site, gRNA scaffold containing MS2 binding sites at stem-loop 2 and tetraloop structures and a zeomycin resistant gene driven by EFalpha promoter (pLV-sgRNA MS2-Zeo) was generated by golden gate assembly of each module. Individual gRNAs were then cloned using BbsI sites in pLV-sgRNA-Zeo. All nine individual gRNAs were also assembled by Golden Gate assembly method. Primer sequences used for Golden Gate assembly (Engler 2009) used BsmBI sites and subcloned into the parental pLV-sgRNA-Zeo to generate pLV-multi gRNA-Zeo. Human codon optimized AR301 LC-T2A-AR301 HC-P2A-Csy4-Zeocin or Csy4-T2A-Zeocin were synthesized de novo and subcloned in pLV-multi gRNA-Zeo to generate a multiplex construct containing or pLV-AR301-Csy4-multi gRNA-Zeo (FIG. 2B, top) or pLV-Csy4-multi gRNA-Zeo (FIG. 2B, bottom).

Production and titer of ssAAV2 containing HDR donor template. ssAAV2 was produced in fast-growing 293FT cells (Invitrogen) by co-transfection of AAV HDR plasmid and AAV packaging helper plasmids (Takara Bio). AAV2 was harvested and purified using AAVpro purification kit (Takara Bio) and titered by real-time PCR.

Reverse transcription real-time quantitative polymerase chain reaction (RT-qPCR). Total mRNA was isolated from 1×10⁶ cells and purified by Zymo's Quick-RNA miniprep kit (Zymo Research) or Trizol and purified using PureLink RNA Mini Kit following manufacturer's instructions (Invitrogen). One-Step RT-qPCR was performed using One-step PrimeScript RT-PCR kit for real-time RT-PCR (Takara Bio) on Applied Biosciences HT7500 real-time PCR machine. 100 ng of total RNA was used per reaction and normalized to mRNA expression of housekeeping genes: GAPDH or EIF3I. Quantification was by standard ddCt method.

Method of engineering myeloma fusion partner cell line with MTRE activation for generation of hybridomas (the myeloma fusion partner cell line is fused with primary B cells to generate an immortalized cell producing an antibody of interest).

Fusion partner cell has several advantages:
Utilizes human primary antibody producing B cells
Bypasses recombinant steps
Little to no process development of the resultant hybridoma needed
Fastest route to clinical manufacturing

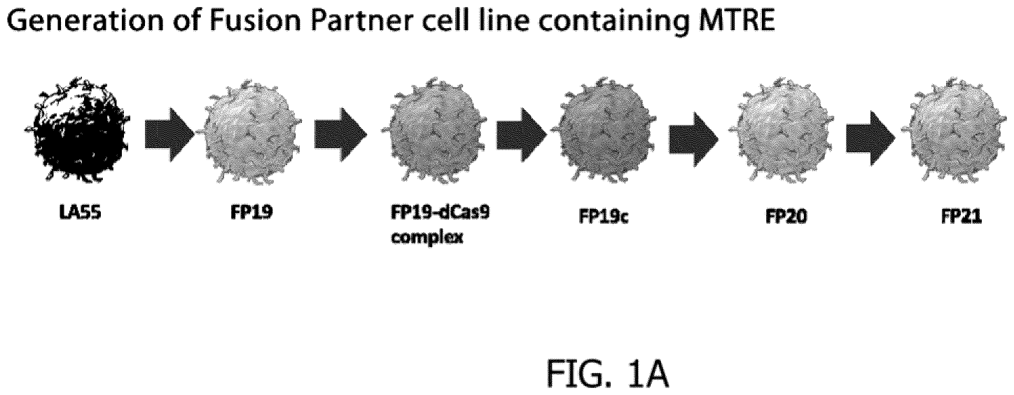

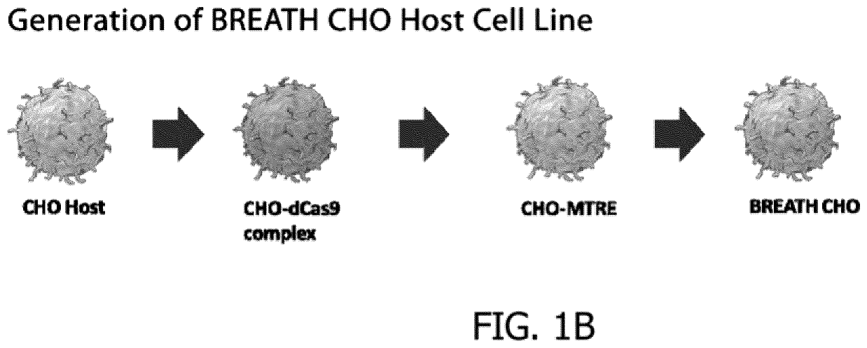

Method of engineering a stable fusion partner cell line containing MTRE activation without native mAb heavy and light chain genes (FP20) and with native mAb heavy and light chain genes (FP21) is outlined in FIG. 1A and Table 1 below:

TABLE 1
Generation of fusion partner cell lines with MTRE activation

| Name | Generation | Method |
|---|---|---|
| LA55 | Parental cell | Parental cell line. |
| | line | |
| FP19 | derived from | Single cell cloning using limited dilution method of single cell |
| | the parental | cloning and screening for growth and productivity using |
| | cell line | CD138. |
| FP19- | | dCas9-VP64 transduction using lentivirus vector and stable |
| dCas | | selection using Blast. |
| Complex | | |
| FP19c | | Transduction of dCas9-VP64 complex with remaining |
| | | accessory components supporting transcriptional activation: |
| | | MS2-p65-HSF1 (MPH). Stable selection using BLAST/Hygro. |
| | | Stably integrated cells are selected by single cell cloning using |
| | | nanowells and adapted to serum free media conditions. |
| FP20 | | Lentivirus mediated transduction of multiplex gRNA (total of |
| | | 9 gRNAs) with stable selection using Blast/Hygro/Zeocin. |
| FP21 | Fusion partner | Lentivirus mediated transduction of protein of interest. For |
| | cell line | antibody production, the transduction of IgGH/IgGL chain |
| | | genes was performed. |

Generation of LA55, a murine-human heteromyeloma cell line: The heteromyeloma cell line LA55 was obtained after fusion of peripheral blood lymphocytes (PBL) of a healthy donor with the hypoxanthine-aminopterin-thymidine (HAT) sensitive murine myeloma cell line X63Ag8.653 (ATCC Cat. #CRL1580). The fusion of somatic cells of different species produces hybrids which usually lose components of chromosomes of the parental species with prolonged subculture. It is known that the chromosomes of the human cells of the parent of a human-murine hybrid cell are gradually lost over successive generations. Therefore, the production of the cell line involved irradiation of the murine myeloma cells to damage some of the murine chromosomes before fusion.

The X63-Ag8.653 murine myeloma cells were irradiated by exposure to 131 rad for 1 minute (in Hanks balanced salt solution, at a concentration of 1×107 cells/ml) before fusion with human B cells. Human B cells were isolated from a peripheral blood sample by gradient centrifugation. Cell fusion was carried out according to standard procedures. Briefly, myeloma cells (4×107) and B cells (1×107) were fused with 50% (wt/vol) polyethylene glycol 4000, and selection was carried out in the presence of HAT and ouabain. Resultant clones were selected for further study based on growth rate and stable secretion of human immunoglobulin heavy and light chains. These clones included a HAT-sensitive, non-secretor clone called LA55 (initially producing human IgM/lambda) was derived by selection in 8-azaguanine and cloning under limiting dilution. In test fusions, LA55 cells consistently demonstrated high fusion efficiency compared with other hybrid clones and to the parental murine myeloma cell line. LA55 is routinely cultured in Iscove's modified Dulbecco's medium (IMDM) supplemented with 10% fetal bovine serum. LA55 was confirmed to be negative for adventitious agents and was used for generation of the engineered fusion partner cell line (FIG. 1A).

Sequential Transduction of FP19 to Generate FP20—Clonal Selection for LA55 Cells with High Expression of CD138:

Single cell clones of LA55 were generated by selection based on expression of murine CD138 by flow cytometry, followed by limiting dilution. Lead clones were selected based on the following criteria:
Longer doubling time: 16-18 hours compared to the original 12 hours.
Homogenous expression of murine CD138.
High fusion efficiency.

Two additional rounds of single cell cloning performed by limiting dilution following lead clone selection was performed (LA55K). For limited dilution cloning, LA55 was seeded in 96-well plates at 0.7 cells/well. Single cell clones were characterized by flow cytometry for homogeneous murine CD138 expression. LA55K cells were then adapted to serum-free conditions using Hybridoma-SFM medium following manufacturer's instructions and renamed as FP19.

FP19-dCas9 complex cells were generated by transduction of lentivirus expression of dCas9-VP64 and stable pool was selected using Blast. FP19c was generated by transduction and stable selection of MPH into FP19-dCas9 cells. FP19-dCas9 was transduced using the same transduction and selection procedures as described above using Blast and Hygro selection. Final Blast and Hygro concentrations used were 20 μg/ml and 400 μg/ml respectively. Stable expression of dCas9-VP64 and MPH was confirmed and expression over 20 generations is demonstrated (FIG. 3).

Lentiviral mediated transduction and stable selection of multi-guide gRNAs activating transcription factors PRDM1, IRF4 and XBP1 (SEQ ID 6-14) into FP19c cells: Multiplex constructs containing gRNAs targeting region within 200 bp of TSS were transduced into FP19c to generate FP20 cell line using methods described above and selected for Blast, Hygro, and Zeocin at 20 μg/ml, 400 μg/ml, and 200 μg/ml. The FP20 cell line therefore contains all the elements of the multi-guide CRISPRa and represents the engineered fusion partner cell line. Multiplex transduction is demonstrated to induce synergistic activation of each transcription factor where the expression of each transcription factor is greater in combination compared to the single transgene (FIG. 4).

Multiplex constructs containing gRNAs targeting the region within 200 bp of the transcription start site (TSS) of MTRE and heavy chain (HC)/light chain (LC) expression of AR-301, a monoclonal antibody (mAb) specific for alpha-toxin of S. aureus (FIG. 2B, top panel), were transduced to FP19c to generate the FP21 host cell line.

In a preferred embodiment, this invention refers to a method of engineering stable host CHO or NS0 cell line containing native mAb H&L chain genes containing Cas9-driven transcriptional activation of MTRE genes Prdm1, Irf4 and Xbp1 and the use of Cas9 editing for site specific replacement of endogenous antibody CDR genes.

BREATH CHO is a CHO host cell line engineered with Cas9-driven transcriptional activation of MTRE genes, Prdm1, Irf4 and Xbp1 were generated in the order as described in FIG. 1B and Table 2 below. Methods of transduction and stable cell line selection were similar to generation of FP19-derived cell lines. We used CHO DG44 host cell line.

CHO DG44 cell line origin: CHO-DG44 in which CHO cells were mutagenized with gamma radiation to yield a cell line in which both alleles of the DHFR locus were completely eliminated, termed CHO-DG44. These DHFR-deficient strains require glycine, hypoxanthine, and thymidine for growth (Urlaub et al., 1983). As the final step, BREATH CHO host cell line was generated by transfection of plasmid DNA expressing heavy chain and light chain genes of AR301 mAb (AR301 HC; AR301 LC).

TABLE 2
Generation of BREATH CHO host cell line.

| Name | Generation | Method |
|---|---|---|
| CHO | Parental cell | Parental cell line may include CHO-DG44, CHOZN, CHO- |
| | line | K1, CHO-S and other CHO derivatives. |
| CHO- | | dCas9-VP64-MPH transduction using lentiviruses and stable |
| dCas | | selection using Blast/Hygro. |
| Complex | | Single cell cloning using limited dilution method of single |
| | | cell cloning and screening for expression of the complete |
| | | cassette of transgene components. |
| | | Stably selected single cell clones were adapted to serum free |
| | | media conditions. |
| CHO- | | Lentivirus mediated transduction of multiplex gRNA (total of |
| MTRE | | 9 gRNAs) with stable selection using Blast/Hygro/Zeocin. |
| BREATH- | | Transfection and stable selection of AR301 HC and AR301 |
| CHO | | LC vectors into CHO-MTRE. |

In another preferred embodiment, this invention refers to the method of enhancing mAb production by Cas9-mediated transcriptional activation of MTRE genes Prdm1, Xbp1 and Irf4 in CHO (CHO-A) or NS0 cell lines already expressing a monoclonal antibody.

Method of Engineering CHO Cell Line with MTRE Activation

Stable CHO cell lines expressing monoclonal antibodies AR-301 or AR-105 were generated by transduction with lentivirus constructs containing dCas9-VP64, accessory proteins (MS2-p65-HSF1) and multiplex guide RNA (SEQ ID 15-25) as described above to increase antibody production in CHO cell lines for Blast, Hygro and Zeo at 20 μg/ml, 200 μg/ml and 20 μg/ml, respectively.

### Method of UPR Activation in CHO Cell Lines

In a further preferred embodiment, this invention refers to a method to temporally induce UPR activation using a small molecule in CHO or NS0 cell lines.

Background: UPR is activated by three separate kinases that controls distinct mechanisms: IRE1, ATF6, and PERK (Walter and Ron, 2011). Activation of UPR can deleteriously impact cell survival. As cell viability is critical in the manufacturing process of recombinant antibody production, we utilized a form of the IRE1 and PERK that does not activate the apoptotic signaling pathway, IRE1(1642G) and FV2E-PERK respectively.

XBP1 functions at the post-translation level to regulate the Unfolded Protein Response (UPR) to expand the ER for protein biosynthesis. In CHO, activation of XBP1 can increase production of recombinant antibody.

Drug-inducible Pseudokinase IRE1 (1642G) mutation by 1NM-PP1 treatment was evaluated. The use of an adenosine triphosphate (ATP) analog, 1NM-PPI to attenuate Ire1 RNase activity via selective binding to the ATP pocket of the kinase domain of IRE1 (1642G) without affecting cell survival (Lin et al., 2007). FV2E-PERK is an artificial PERK allele that can dimerize with the small molecule, AP20187 in CHO cells to active PERK signaling (Lu et al., 2004).

In a preferred embodiment, this invention refers to dosage dependent UPR activation by a small molecule drug, 1NM-PP1 wherein when induced in stable CHO cell line that expresses Ire1-1642G in combination with Fv2E-PERK under the control of constitutive promoters.

Vector construction: Coding region of humanized Ire1a 1642G were codon-optimized for Chinese hamster ovary (CHO), synthesized de novo and subcloned at the BsiWI/EcoRI cloning site of the pLV-dCas9-VP64-Bsd vector. For construction of Fv2E-PERK, kinase domain of PERK was fused to two modified FK506 binding domains and codon optimized for CHO cell expression. Lentiviral vectors containing Ire1a 1642G and Fv2E-PERK are shown in FIG. 12.

Lentiviral vector expressing Ire1(1642G) and Fv2E-PERK: Coding regions for Ire1(1642G) and Fv2E-PERK were placed in-frame flanked by 2A self-cleaving peptide from Thosea asigna virus. Combined fragment was cloned in lentiviral vector containing EF1A promoter and Hygromycin resistant gene.

Stable cell line expressing either Ire1a 1642G or Fv2E-PERK alone or in combination were engineered by lentivirus transduction and selection of Blast or Hygro resistant-cell lines (see above).

1NM-PP1 drug induction in CHO cells: Stable CHO cell lines expressing Ire1a 1642G or Fv2E-PERK alone or in combination were induced with 10 or 50 UM of 1NM-PP1. Gene expression measured by RT-qPCR was performed at 24h post-drug induction.

In a preferred embodiment, the invention refers to site specific replacement of endogenous antibody CDR genes by CRISPR enzyme editing of a monoclonal antibody production CHO host cell line containing Cas9 or Cpf1 gene activation of MTRE genes PRDM1, XBP1, IRF4. CRISPR enzymes include Cas9, Cpf1, CasX, CasY, Cas13, or Cas14.

### Method of Gene Swap of Heavy and Light Chain Genes in mAb Producing Cell Line Using CRISPR

Background: In biomanufacturing, process development is required for every mAb and includes both upstream and downstream process development (Gronemeyer, 2014) to produce an antibody product. Areas for improvement of antibody yield can include process efficiency, or high cell production density to improve the efficiency of cell culture processes. Antibody concentrations as high as 10-13 g/L have been achieved in fed-batch processes (Li, 2010).

Antibodies (Abs) are formed by heavy and light chains composed of constant and variable regions where they bind their targets using highly diversified loops, termed complementarity-determining regions (CDRs), with three in each rearranged VH and VL gene. CDRs 1 and 2 are encoded by germline V genes, while CDR3s in both VH and VL are the product of gene recombination. The varied length and biochemical properties of heavy-chain and light-chain complementarity-determining region 3 (CDR-H3 and CDR-L3) contribute to enhanced sequence diversity (D'Angelo, 2018). Theoretical CDR-H3 diversity can exceed 1015 variants. The human antibody repertoire is highly diverse, with the incorporated CDR-H3 derived from the sequential assembly of 56 VH, 23 DH, and 6 JH genes. It is clear that CDR-H3 and CDR-L3 is the major determinant of antibody-binding specificity (Mahon et al., 2013; Shirai 1996), such that exchanging the CDR3 region from a cell line containing native framework region of a human mAb is sufficient for generation of a new mAb produced by CHO or NS0 because the antigen specificity is determined by their CDR-H3 and CDR-L3 domains. The methods described here utilizes Cas9 technology to exchange the CDR3 of a master production cell line to produce a new cell line of alternate antibody specificity with the benefit of leveraging the defined process development of the production master cell line and reducing extensive process development.

Ribonucleoprotein complex formation. Single guide RNA (sgRNA) without PAM site for Streptococcus pyrogenes Cas9 (SpCas9) were designed and synthesized by Synthego. These sgRNA target 20 nt DNA sequence at the 5′ and 3′ end of AR-301 CDR-H3 and CDR-L3. For CDR3 switching, 5 sgRNA candidates targeting the end of FR3 and beginning of FR4 region for CDR-H3 switching; and similar approach was used to select gRNAs for CDR-L3 switching. sgRNA combined with purified SpCas9 protein were allowed to complex by heating at 95° C. followed by cooling to room temperature prior to RNP delivery by electroporation.

Vector construction of homology-directed repair (HDR) donor template for CDR3 switching. Zsgreen1 cassette (containing stop codon, TGA, followed by Zsgreen1 cassette containing CMV promoter, coding region of Zsgreen1 fluorescent protein and SV40 polyA tail) were generated by de novo synthesis and further subcloned into a high-copy pUC19 plasmid (NEB) using In-Fusion snap assembly (Takara Bio). 5′ and 3′ homology arms (HA) targeting 750 bp DNA regions at the 5′ and 3′ regions of CDR-H3 and CDR-L3 (see FIG. 13B) were synthesized de novo. Final HDR donor template was constructed by In-Fusion snap assembly of the following fragments from 5′ to 3′ into single stranded AAV (ssAAV) vector downstream of CMV promoter: 750 bp 5′ HA, Zsgreen1 coding region and 750 bp 3′ HA. Production and titer of ssAAV2 containing HDR donor template are described above.

RNP delivery. RNP was delivered by electroporation into CHO DG44 cells using Neon transfection system according to manufacturer's recommendations (see above). Following RNP delivery, cells were transduced using ssAAV2 at a MOI of 1×10⁵ viral genome/cell. 48 hours post-transduction, cells were seeded onto nanoarrays and fluorescence and secretion levels were measured using IgG diffusion assay (Bobo et al., 2014).

In another preferred embodiment, this invention refers to the method of constructing a high productivity monoclonal antibody production murine heteromyeloma fusion partner cell line containing dCas9 or dCpf1 gene activation of MTRE genes PRDM1, XBP1, IRF4 and the use Cas9 editing for site specific replacement of endogenous antibody H&L genes the same as in CDR switching, as discussed above with modifications to vector construction of HDR donor and sgRNA targeting regions as described below.

Ribonucleoprotein complex formation. Single guide RNA (sgRNA) without PAM site for Streptococcus pyrogenes Cas9 (SpCas9) were designed and synthesized by Synthego. These sgRNA target 20 nt DNA sequence downstream of CAG promoter of pCAG-AR301 HC-pA vector (SEQ ID 26-31) and upstream of SV40 poly A tail 5′ and 3′ end of AR-301 H&L-H3 and CDR-L3 (SEQ ID 32-49). sgRNA combined with purified SpCas9 protein were allowed to complex by heating at 95° C. followed by cooling to room temperature prior to RNP delivery by electroporation.

Vector construction of homology-directed repair (HDR) donor template for H&L switching. 5′ and 3′ homology arms (HA) targeting 750 bp DNA regions downstream of CAG promoter of pCAG-AR301 HC-pA vector and upstream of SV40 poly A tail (3′ HA) were synthesized de novo. Final HDR donor template was constructed by In-Fusion snap assembly of the following fragments from 5′ to 3′ into single stranded AAV (ssAAV) vector downstream of CMV promoter: 750 bp 5′ HA, Zsgreen1 coding region and 750 bp 3′ HA.

RNP delivery. RNP was delivered by electroporation into CHO DG44 cells using Neon transfection system according to manufacturer's recommendations (see above). Following RNP delivery, cells were transduced using ssAAV2 at a MOI of 1×10⁵ viral genome/cell. 48 hours post-transduction, cells were seeded onto nanoarrays and fluorescence and secretion levels were measured using IgG diffusion assay (Bobo et al., 2014).

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Stable Expression of Transgenes in Fusion Partner Cells (FP19c)

Stable cell lines of FP19, FP19-dCas9, and FP19c were analyzed for expression of dCas9-VP64 and MS2-p65-HSF1 by RT-qPCR. Expression of dCas9-VP64 and MS2-p65-HSF1 transgenes were measured in comparison to FP19 at generations 1, 10, and 20 as described above. FP19c cells were evaluated for expression of dCas9-VP64 and MPH transgenes and confirmed to have similar levels of each transcript by qRT-PCR. CRISPR-mediated gene transduction of FP19c cells was confirmed to have stable expression of transgenes dCas9-VP64 and MPH over 20 generations (FIG. 3).

### Example 2. Synergistic Activation of MTRE Using Multiplex sgRNA Approach

FP19 and FP19c were transfected with pLV-Csy4-multi gRNA-Zeo or pLV-sgRNA-Zeo containing target sequences for PRDM1, IRF4, and XBP1 promoter regions). RT-qPCR using primers for human PRDM1, IRF4, and XBP1 were performed as described above. The multiplex approach to TF activation showed higher transcriptional activation of transcription factors compared to single gRNA activation consistent with synergy as demonstrated in FIG. 4.

### Example 3. Proof of Activation of MTRE and Enhanced IgH and IgL Transcription Using Multiplex sgRNA Approach in Stable Fusion Partner Cell Line, FP21

FP21 is a stable cell expressing dCas9-VP64, MPH, and multiplex gRNA targeting promoter regions of human PRDM1, IRF4, and XBP1 promoter regions. FP-AR301 was generated by lentivirus transduction and stable selection of cassette expressing AR-301 heavy chain and AR-301 light chain genes. RT-qPCR using primers for human PRDM1, IRF4, and XBP1 were performed as described above. FP-AR301 MTRE showed enhanced transcription of PRDM1, IRF4 and XBP1 compared to FP-AR301 cell line (FIG. 5A). mRNA transcripts of heavy and light chain genes of immunoglobulin (IgH and IgL, respectively) were measured by RT-qPCR for FP-AR301 and FP-AR301 MTRE (FIG. 5B). FP-AR301 MTRE shows enhanced expression of IgH and IgL transcripts compared to FP-AR301 (FIG. 5B).

### Example 4. Increase in Antibody Production Following MTRE Activation in Fusion Partner Host Cell Lines

Antibody production was measured in FP-AR301 (without MTRE activation) and FP-AR301 MTRE (with MTRE activation) by 10-day batch study in T75 flasks. Supernatants were collected at Day 0 and Day 10 and IgG titer was measured by human IgG sandwich ELISA (FIG. 6).

### Example 5. Increase in MTRE Expression Following CRISPR-Mediated MTRE Activation in CHO Host Cell Lines

CRISPR mediated activation of MTREs in CHO-DG44 (CHO MTRE) cell line demonstrates marked fold change in transcript levels as measured by qPCR. Increased mRNA transcripts of dCas9 complex and MPH are shown. Increased transcripts of MTREs PRDM1, XBP1, and IRF4 by RT-qPCR are demonstrated. Fold change expression relative to CHO DG44 host cell line without AR-301 antibody expressing genes was calculated based on 2-44Ct (FIG. 7A). Transcript levels were normalized to EIF3I. In the present example, there is a marked increase in IgH and IgL transcription in BREATH CHO DG44 cell line (FIG. 7B).

### Example 6. Antibody Production of Engineered BREATH CHO Host Cell Line

Antibody production of AR-301 mAb was measured in BREATH CHO by 10-day batch study in shake flask. Supernatants were collected at Days 0, 2, 5, 7, and 9 and cumulative and IgG titer was measured by human IgG sandwich ELISA. Endpoint titer for BREATH CHO DG44 at Day 10 was determined to be 0.3 g/L (FIG. 8).

### Example 7. Increase in Antibody Production Following MTRE Activation in CHO Cell Lines Expressing AR-105

MTREs activation in stable cell line expressing AR-105 mAb (CHO-A AR105) was measured by RT-qPCR looking at mRNA transcripts of Prdm1, Xbp1, and Irf4 in Chinese hamster ovary cells. Compared to original CHO-AR105 (without MTRE activation), CHO-A AR105 shows enhanced transcription of each MTRE (Prdm1, Xbp1, and Irf4, FIG. 9A). Following comparison of MTRE transcription in CHO-A AR105 and CHO-AR105, we examined IgG secretion level of 10,000 single cell clones of CHO-A AR105 and CHO-AR105 on nanowells. On a population level, CHO-A AR105 shows a two-fold enhanced fluorescence level (FIG. 9B-C), indicative of secretion level compared to CHO-AR105. AR 105 is a human monoclonal antibody that binds to mucoid exopolysaccharide (MEP) of P. aeruginosa.

### Example 8. Increase in Antibody Production Following MTRE Activation in CHO Cell Lines Expressing AR-301

MTREs activation in multiple stable cell line expressing AR-301 mAb (CHO-A AR301) was measured by RT-qPCR looking at mRNA transcripts of Prdm1, spliced Xbp1 (Xbp1s), and Irf4 in Chinese hamster ovary cells. Xbp1s is a more accurate measure of Xbp1 activity. Compared to original CHO-AR301 (without MTRE activation), CHO-A AR301 shows enhanced transcription of each MTRE (Prdm1, Xbp1s, and Irf4, FIG. 10A) as well as enhanced IgH and IgL transcription, measured by RT-qPCR (FIG. 10B). Following comparison of MTRE, IgH and IgL transcription in CHO-A AR301 and CHO-AR301, we examined IgG secretion level of two clones in T75 flask (FIG. 10C). CRISPR-mediated MTRE activation in CHO-AR301 (CHO-A AR301) shows enhanced secretion compared to without MTRE activation (CHO-AR301).

### Example 9. Drug-Inducible UPR Activation of CHO Cell Lines by Constitutive Expression of Ire1-1642G and Fv2E-PERK Combined with 1NM-PP1

A drug inducible UPR activation pathway was constructed in a CHO-K1 cell line transfected with plasmid containing constitutive promoter expressing Ire1-1642G or Fv2E-PERK alone or in combination. At 24 hours post-transfection, cells were treated with varying concentrations of 1NM-PP1 (0, 10 or 50 μM). mRNA transcripts of UPR genes were measured by RT-qPCR of unspliced Xbp1 (Xbp1u), spliced Xbp1 (Xbp1s), Chop, Gadd34, and Atf4. Synergy is observed when Ire1-1642G and Fv2E-PERK expressed in combination in a dosage-dependent manner where transcripts of Xbp1u, CHOP, Gadd34, and Atf4 are increased at higher concentrations of 1NM-PP1 (FIG. 12A).

Inducible UPR pathway activation in stable cell line expressing AR-301 mAb was generated by co-transfection of plasmids containing constitutive promoter expressing Ire1-1642G and Fv2E-PERK (+UPR) or mock (−UPR; FIG. 12B). At 24 hours post-transfection, cells were treated with 10 UM of 1NM-PP1. After 24 hours post-treatment of 1NM-PP1, CHO-AR301 cells with and without UPR activation were seeded at the same cell density (3E5 viable cells/ml) in 20 ml seeding volume for shake flask production run for 7 days. Secreted IgG protein levels of AR-301 mAb at Day 7 with and without UPR activation were measured by IgG sandwich ELISA.

### Example 10. Switching of CDR-H3 and CDR-L3 into CHO Host Cell Lines by CRISPR Directed Homologous Recombination

A ribonucleoprotein (RNP) complex consisting of Cas9 and sgRNA are assembled in vitro. The sgRNA targets 5′ and 3′ end of AR-301 CDR-H3 and CDR-L3. Following RNP entry into the cell by electroporation and delivery of ssDNA of HDR donor template, editing occurs via homology directed recombination (HDR). The cell with CDR3 switched to Zsgreen1 cassette is shown by the presence of green fluorescence signal but no secreted antibody signal (FIG. 13D, measured by red fluorescence, red). Detection of successful gene swap was performed at 72h following RNP delivery. The edited CHO cells were seeded in nanowells coated with anti-human IgG capture antibody. Secondary detection antibody conjugated to Cy3 was added to detect for clones secreting the parental full-length heavy and light chain antibody genes of AR-301. Images were acquired at 24 hours post-seeding. Bottom left panel shows 60,000 nanowells while bottom right panel is a magnified inset showing successful switching of CDR-H3 and CDR-L3 to Zsgreen1 cassette.

### Example 11. Switching of Heavy and Light Chains in CHO Host Cell Lines by CRISPR Directed Homologous Recombination

The ribonucleoprotein (RNP) complex consisting of Cas9 and sgRNA was assembled in vitro. The sgRNA targets the 5′ and 3′ ends of the AR-301 HC and LC. Following RNP entry into the cell by electroporation and delivery of ssDNA of HDR donor template, editing occurs via homology directed recombination (HDR). Edited CHO cells are selected for the presence of ZsGreen1 fluorescence and absence of IgG secretion (FIG. 11E, measured by red fluorescence, red). The bottom left panel shows 60,000 nanowells while the bottom right panel is a magnified inset showing successful switching of AR-301 mAb H&L to Zsgreen1 cassette.

### REFERENCES

Bobo B, Phelan D, Rebhahn J, et al. Microbubble array diffusion assay for the detection of cell secreted factors. Lab Chip.; 14(18):3640-3650, 2014.
Cho S, Shin J, Cho B K. Applications of CRISPR/Cas System to Bacterial Metabolic Engineering. Int J Mol Sci. April 5; 19(4):1089, 2018.
Kohler, G., Milstein, C. Continuous cultures of fused cells secreting antibody of predefined specificity. Nature 256, 495-497, 1975.
Donillard and Hoffinan: Compendium of Immunology V, article “Basic Facts about Hybridomas” 1981.
D'Angelo S, Ferrara F, Naranjo L, Erasmus M F, Hraber P, Bradbury ARM. Many Routes to an Antibody Heavy-Chain CDR3: Necessary, Yet Insufficient, for Specific Binding. Front Immunol. March 8; 9:395, 2018.
Dangi A K, Sinha R, Dwivedi S, Gupta S K, Shukla P. Cell Line Techniques and Gene Editing Tools for Antibody Production: A Review. Front. Pharmacol. 9:630, 2018.
Gronemeyer P, Ditz R, Strube J. Trends in Upstream and Downstream Process Development for Antibody Manufacturing. Bioengineering (Basel). October 1; 1(4):188-212, 2014.
Koch H. Urwyler S, Rudolf M, Truong V, 2016, Human Monoclonal antibody specific for the F protein of Respiratory Syncytial Virus (RSV), U.S. Patent No. WO2016069904 Washington, D C: U.S. Patent and Trademark Office.
Konermann S, Brigham M D, Trevino A E, Joung J, Abudayyeh O O, Barcena C, Hsu P D, Habib N, Gootenberg J S, Nishimasu H, Nureki O, Zhang F. Genome-scale transcriptional activation by an engineered CRISPR-Cas9 complex. Nature. January 29; 517(7536):583-8, 2015.
Li F, Vijayasankaran N, Shen A Y, Kiss R, Amanullah A. Cell culture processes for monoclonal antibody production. MAbs.; 2(5):466-479, 2010.
Lin J H, Li H, Yasumura D, et al. IRE1 signaling affects cell fate during the unfolded protein response. Science.; 318(5852):944-949, 2007.
Lu P D, Jousse C, Marciniak S J, et al. Cytoprotection by pre-emptive conditional phosphorylation of translation initiation factor 2. EMBO J.; 23(1):169-179, 2004.
Mahon C M, Lambert M A, Glanville J, Wade J M, Fennell B J, Krebs M R, Armellino D, Yang S, Liu X, O'Sullivan C M, Autin B, Oficjalska K, Bloom L, Paulsen J, Gill D, Damelin M, Cunningham O, Finlay W J. Comprehensive interrogation of a minimalist synthetic CDR-H3 library and its ability to generate antibodies with therapeutic potential. J Mol Biol. May 27; 425(10):1712-30, 2013.
Martincic K, Alkan S A, Cheatle A, Borghesi L, Milcarek C. Transcription elongation factor ELL2 directs immunoglobulin secretion in plasma cells by stimulating altered RNA processing. Nat Immunol. October; 10(10): 1102-9, 2009.
Pier, G, Preston M, Cavacini L, Posner M, P. aeruginosa mucoid exopolysaccharide specific binding peptides. U.S. Patent No. US20030124631A1 Washington, DC: U.S. Patent and Trademark Office. 2003.
Rudolf M, Koch H. Human monoclonal antibody against S. aureus derived alpha-toxin and its use in treating or preventing abcess formation. World Intellectual Property Organization Publ. of the Int. Appl. with Int. search report WO2010EP04884, 2010.
Sciammas R, Davis M M. Modular nature of Blimp-1 in the regulation of gene expression during B cell maturation. J Immunol. May 1; 172(9):5427-40, 2004.
Shaffer A L, Shapiro-Shelef M, Iwakoshi N N, Lee A H, Qian S B, Zhao H, Yu X, Yang L, Tan B K, Rosenwald A, Hurt E M, Petroulakis E, Sonenberg N, Yewdell J W, Calame K, Glimcher L H, Staudt L M. XBP1, downstream of Blimp-1, expands the secretory apparatus and other organelles, and increases protein synthesis in plasma cell differentiation. Immunity. July; 21(1):81-93, 2004.
Shapiro-Shelef M, Calame K. Regulation of plasma-cell development. Nat Rev Immunol. March; 5(3):230-42, 2005.
Shirai H, Kidera A, Nakamura H. Structural classification of CDR-H3 in antibodies. FEBS Lett. December 9; 399 (1-2): 1-8, 1996.
Swaminathan, S., Klemm, L., Park, E. et al. Mechanisms of clonal evolution in childhood acute lymphoblastic leukemia. Nat Immunol 16, 766-774, 2015.
Tellier J, Shi W, Minnich M, Liao Y, Crawford S, Smyth G K, Kallies A, Busslinger M, Nutt S L. Blimp-1 controls plasma cell function through the regulation of immunoglobulin secretion and the unfolded protein response. Nat Immunol. March; 17(3):323-30, 2016.
Urlaub G, Käs E, Carothers A M, Chasin L A. Deletion of the diploid dihydrofolate reductase locus from cultured mammalian cells. Cell. June; 33(2):405-12, 1983.
Walter P, Ron D. The unfolded protein response: from stress pathway to homeostatic regulation. Science. November 25; 334(6059):1081-6, 2011.
Zhu, H., Bhatt, B., Sivaprakasam, S. et al. Ufbp1 promotes plasma cell development and E R expansion by modulating distinct branches of UPR. Nat Commun 10, 1084, 2019.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An antibody production system comprising:
a cell line producing an antibody of interest, wherein one or more cells of the cell line comprise:
CRISPR/dCas9 or CRISPR/Cas12a linked to a transcriptional activator;
a first guide RNA (gRNA) having a spacer sequence complementary to promoter of a first transcription factor; and,
a second gRNA having a spacer sequence complementary to the promoter of a second transcription factor;
wherein the first and second gRNAs are different from each other and the first and second transcription factors are different from each other; and,
wherein the transcription factors are associated with expression of the antibody of interest.

2. The system of claim 1, wherein the cell line is selected from the group consisting of: CHO, NS0, Sp2/0, Vero, HEK 293, or 293T, and murine myeloma cell line X63Ag8.653.

3. The system of claim 1, wherein the transcriptional activator is selected from the group consisting of: VP64, VP16, and activation helper protein complex MS2-P65-HSF1 (MPH.)

4. The system of claim 1, wherein the first and second transcription factors are selected from the group consisting of PRDM1 (Blimp1), XBP1, and IRF4.

5. The system of claim 1, wherein the one or more cells further comprise a third gRNA having a spacer sequence complementary to the promoter of a third transcription factor different from the first and second transcription factors.

6. The system of claim 5, wherein the first and second transcription factors are selected from the group consisting of XBP1, IRF4, PRDM1, ELL2, SPI1, pTEFb, AFF4, AFF1, SUPT5HM, DOT1L, IRE1, PERK, BIP, SRP9, SRP14, and SRP54.

7. The system of claim 1, wherein the one or more cells further comprise an inhibitor to expression of proteins selected from the group consisting of: AICDA, SIAH1, and HNRNP F.

8. The system of claim 1, wherein the antibody production system transcription factors are endogenous to the one or more cells.

9. The system of claim 1, wherein one or more of the antibody production system transcription factors are encoded by recombinant nucleic acids present within the one or more cells.

10. The system of claim 1, further comprising one or more additional gRNAs complementary to promoter of the first or second transcription factor at a position different from that of the first or second gRNA.

11. The system of claim 10, wherein there are two or more gRNAs with different spacer sequences directed to the same promoter or downstream promotor elements (DPE).

12. The system of claim 1, wherein expression enhancement is accomplished using a single gRNA directed to the promoter for PRDM1, IRF4, or XBP1.

13. An antibody production system comprising:
a cell line producing an antibody of interest, wherein one or more cells of the cell line comprise:
CRISPR/dCas9 linked to a transcriptional activator selected from the group consisting of VP64, VP16, and activation helper protein complex MS2-P65-HSF1 (MPH);
one or more first gRNAs having a spacer sequence complementary to the promoter of PRDM1 or to the promoter of a peptide downstream in a pathway from PRDM1;
one or more second gRNAs having a spacer sequence complementary to the promoter of XBP1; and,
one or more third gRNAs having a spacer sequence complementary to the promoter of IRF4;
wherein the cell line is selected from the group consisting of CHO, NS0, Sp2/0, and murine myeloma cell line X63Ag8.653;
wherein the transcriptional activator is active in enhancing expression of the PRDM1, XBP1, or IRF4; and,
wherein the transcription factors are directed to enhance expression of the antibody of interest.

14. The system of claim 13, wherein the cells comprise the transcription factor sequences both genome-integrated and as extra-genomic nucleic acids.

15. The system of claim 13, wherein the cells comprise extra-genomic nucleic acids encoding the antibody of interest.

16. A method of enhancing expression of an antibody of interest, the method comprising:
providing a cell comprising a nucleic acid having a sequence encoding a heavy chain of the antibody and a nucleic acid having a sequence encoding the light chain of the antibody;
providing in the cell a CRISPR/dCas9 linked to a transcriptional activator;
providing in the cell a first guide RNA (gRNA) having a spacer sequence complementary to the promoter or DPE of a first transcription factor; and
providing in the cell a second gRNA having a spacer sequence complementary to the promoter of a second transcription factor;
wherein the first and second gRNAs are different from each other and the first and second transcription factors are different from each other; and,
wherein the transcription factors are associated with enhanced expression of the heavy chain and light chain of antibody of interest

17. The method of claim 16, further comprising one or more additional gRNAs complementary to the first or second promoter but at a different part of the promoter.

18. The method of claim 16, wherein:
the CRISPR/dCas9 linked transcriptional activator is selected from the group consisting of VP64, VP16, and activator helper complex MS2-P65-HSF1 (MPH);
the promoter or DPE complementary to the first gRNA controlling expression of PRDM1 or a peptide downstream in a pathway from PRDM1;
the promoter or downstream promoter elements (DPE) controlling expression of XBP1; or,
the cell is provided with a gRNA complementary to the promoter or downstream promoter elements (DPE) controlling expression of IRF4; and,
the cell line is selected from the group consisting of CHO, NS0, Sp2/0, Vero, HEK 293 or 293T and murine myeloma cell line X63Ag8.653.

19. The method of claim 16, further comprising:
providing the CRISPR/dCas9-activator or gRNAs by electroporation, through Lentivirus transfection, or through encoded transposon sequences.

20. The method of claim 16, further comprising providing to the cell the nucleic acid having the sequence encoding a heavy chain of the antibody and a nucleic acid having the sequence encoding the light chain of the antibody as follows:
providing CRISPR/Cas9;
providing a third gRNA with a spacer sequence complementary to a heavy chain sequence or to a heavy chain CDR target sequence endogenous to the cell;
providing a fourth gRNA with a spacer sequence complementary to a light chain sequence or to a light chain CDR target sequence endogenous to the cell;
providing in the cell an editing template of nucleic acid having a desired heavy chain sequence, light chain sequence, heavy chain CDR sequence, or light chain CDR sequence;
the editing template also having adjacent homologous regions complementary to sequences flanking the target sequence;
hybridizing the gRNAs with their complementary target sequences;
cutting the targeted endogenous sequences with the CRISPR/Cas9 at a site of gRNA hybridization;
repairing the cut by homology directed repair (HDR), thereby replacing the endogenous target sequence of the cell with the desired heavy chain sequence, light chain sequence, heavy chain CDR sequence, or light chain CDR sequence;
whereby antibody expression of the cell is converted over from previous endogenous expression to expression of the antibody of interest.

21. The method of claim 20, wherein the CDR is a CDR3.

22. A gRNA multiplex expression system comprising:
a nucleic acid strand encoding two or more gRNA sequences each comprising a different spacer sequence to a different region in promoter or DPE for the same transcription factor,
wherein the promoter or DPE is associated with expression of an antibody.

23. The system of claim 22, wherein the transcription factor is selected from the group consisting of XBP1, IRF4, PRDM1, ELL2, SPI1, pTEFb, AFF4, AFF1, SUPT5HM, DOT1L, IRE1, PERK, BIP, SRP9, SRP14, and SRP54
